(19) United States
(12) Patent Application Publication
(10) Pub. No.: US 2017/0035344 A1
(43) Pub. Date: Feb. 9, 2017

(54) Detection of an Allergic Reaction Using Thermal Measurements of the Face

(71) Applicant: Facense Ltd., Kiryat Tivon (IL)

(72) Inventors: Arie Tzvieli, Berkeley, CA (US); Ari M. Frank, Haifa (IL); Gil Thieberger, Kiryat Tivon (IL)

(73) Assignee: Facense Ltd., Kiryat Tivon (IL)

(21) Appl. No.: 15/231,276

(22) Filed: Aug. 8, 2016

Related U.S. Application Data
(60) Provisional application No. 62/202,808, filed on Aug. 8, 2015.
(60) Provisional application No. 62/236,868, filed on Oct. 3, 2015.

(51) Int. Cl. A61B 5/00; A61B 5/01

(52) U.S. Cl. CPC A61B 5/411; A61B 5/0075; A61B 5/015; A61B 5/6803; A61B 5/0077; A61B 5/7278; A61B 5/7282; A61B 5/0008; A61B 2562/0276; A61B 2576/00

(57) ABSTRACT

Described herein are systems, methods, and computer programs for detecting an allergic reaction. In one embodiment, a system configured to detect an allergic reaction of a user, includes: a frame configured to be worn on the user's head; a thermal camera, weighing less than 5 g, physically coupled to the frame, located less than 10 cm away from the user's face, and configured to take thermal measurements of at least part of the user's nose (TH_{N}); and a circuit configured to determine an extent of the allergic reaction based on TH_{N}.

### TECHNICAL FIELD

This application relates to wearable head-mounted systems that include one or more thermal cameras for taking thermal measurements.

### BACKGROUND

Many physiological responses are manifested in the temperature that is measured on various regions of the human face. For example, facial temperatures may be indicative of the amount of stress a person might be under, whether the person is having an allergic reaction, or the level of concentration the person has at a given time. In another example, facial temperatures can be indicative of a user's emotional state, e.g., whether the user is nervous, calm, or happy.

Thus, monitoring and analyzing facial temperatures can be useful for many health-related and life logging-related applications. However, collecting such data over time, when people are going about their daily activities, can be very difficult. Typically, collection of such data involves utilizing thermal cameras that are bulky, expensive, and need to be continually pointed at a person's face. Additionally, due to the movements involved in day-to-day activities, various image analysis procedures need to be performed, such as face tracking and registration, in order to collect the required measurements.

Therefore, there is a need for way to be able to collect measurements of facial temperatures at various regions of a person's face. Preferably, the measurements need to be able to be collected over a long period of time, while the person performs various day-to-day activities.

### SUMMARY

Various aspects of this disclosure involve head-mounted systems that are utilized to take thermal measurements of a user's face for various applications such as detection of physiological reactions such as an allergic reaction, stress, or various security-related applications. Typically, these systems involve one or more thermal cameras that are coupled to a frame worn on the user's head and are utilized to take thermal measurements of one or more Regions Of Interest (ROIs). The thermal measurements can then by analyzed to detect various physiological reactions. Optionally, the frame may belong to various head-mounted systems, ranging from eyeglasses to more sophisticated headsets, such as virtual reality systems, augmented reality systems, or mixed reality systems.

In different embodiments described herein, one or more thermal cameras are physically coupled to a frame of a head-mounted system (HMS), in such a way, that they remain pointed at the same area on the face (the same ROI) even when the user moves his/her head in angular movements that exceed 0.1 rad/sec. Having the thermal cameras remain pointed at their respective ROIs enables, in some embodiments, to forgo or reduce the need to utilize certain image analysis procedures, such as face tracking and registration, in order to process the collected data.

Various embodiments described herein utilize lightweight thermal cameras, such as thermal cameras that each weigh less than 5 grams or even less than one gram. Optionally, the thermal camera is based on at least one of the following uncooled sensors: a thermopile, a pyroelectric, and a microbolometer.

One aspect of this disclosure involves a system configured to determine an extent of an allergic reaction of a user. In one embodiment, the system includes at least a frame, a thermal camera, and a circuit. The frame is configured to be worn on the user's head and the thermal camera is physically coupled to the frame and located less than 10 cm away from the user's face. The thermal camera, which weighs less than 5 gram, is configured to take thermal measurements of at least part of the user's nose (TH_{N}). Optionally, the thermal camera is based on at least one of the following uncooled sensors: a thermopile, a pyroelectric, and a microbolometer. Optionally, the thermal camera is not in physical contact with the nose, and remains pointed at the nose when the user's head makes angular movements also above 0.1 rad/sec. Optionally, the thermal camera is located less than 3 cm away from the user's face and weighs below 1 g. Optionally, the system does not occlude the ROI. Additional discussion regarding some of the properties of the thermal camera (e.g., accuracy) is given further below. The circuit is configured to determine an extent of the allergic reaction based on TH_{N}. Optionally, determining the extent of the allergic reaction involves determining whether there is an onset of an allergic reaction. Optionally, determining the extent of the allergic reaction involves determining a value indicative of the severity of the allergic reaction. Optionally, the measurements taken by the thermal camera, which are utilized by the circuit to determine the extent of the allergic reaction, may include measurements of regions near the user's mouth (e.g., the lips and/or edges of the mouth).

Another aspect of this disclosure involves a system configured to estimate stress level of a user wearing a head-mounted system (HMS). In one embodiment, the system includes at least a frame, a thermal camera, and a circuit. The frame is configured to be worn on the user's head and the thermal camera, which weighs below 5 g, is physically coupled to the frame and located less than 10 cm away from the user's face. The thermal camera is configured to take thermal measurements of a region of interest (TH_{ROI}), where the ROI covers at least part of the area around the user's nose. Optionally, the thermal camera is located less than 3 cm away from the user's face and weighs below 1 g. Optionally, the system does not occlude the ROI. The circuit is configured to estimate the stress level based on TH_{ROI}. The circuit may be any of the various types of circuits mentioned in this disclosure, e.g., it may be a processor, an ASIC, or an FPGA. In one example, the circuit is the circuit 16 described in FIG. 1a. In some embodiments, the circuit may be coupled to the frame and/or to an HMS of which the frame is a part. In other embodiments, the circuit may belong to a device carried by the user (e.g., a processor of a smartwatch or a smartphone).

Some systems described in this disclosure involve at least two thermal cameras that are used to take thermal measurements of possibly different ROIs. An example of such a system includes at least a frame, a first thermal camera, and a second thermal camera. The frame is configured to be worn on a user's head. The first thermal camera is physically coupled to the right side of the frame and is located less than 10 cm away from the user's face. Herein, “cm” refers to centimeters. The first thermal camera is configured to take thermal measurements of a first region of interest (TH_{ROI1}). Optionally, ROI₁ covers at least a portion of the right side of the user's forehead, and the system does not occlude ROI₁. The second thermal camera is physically coupled to the left side of the frame and is located less than 10 cm away from the user's face. The second thermal camera is configured to take thermal measurements of a second region of interest (TH_{ROI2}). Optionally, ROI₂ covers at least a portion of the right side of the user's forehead, and the system does not occlude ROI₂. Optionally, the system includes a circuit configured to utilize TH_{ROI1} and TH_{ROI2} to detect a physiological reaction such as an allergic reaction or stress.

Some systems described in this disclosure involve at least four thermal cameras that are used to take thermal measurements of possibly different ROIs. An example of such a system includes at least a frame, and first, second, third and fourth thermal cameras. The frame is configured to be worn on a user's head, and the first, second, third and fourth thermal cameras remain pointed at their respective ROIs when the user's head makes angular movements. For example, the first, second, third and fourth thermal cameras may remain pointed at their respective ROIs when the user's head makes angular movements that exceed 0.1 rad/sec. In one embodiment, the first and second thermal cameras are physically coupled to the frame and are located to the right and to the left of the symmetry axis that divides the user's face to the right and left sides, respectively. Additionally, each of these thermal cameras is less than 10 cm away from the user's face.

The first thermal camera is configured to take thermal measurements of a first region of interest (TH_{ROI1}), where ROI₁ covers at least a portion of the right side of the user's forehead. The second thermal camera is configured to take thermal measurements of a second region of interest (TH_{ROI2}), where ROI₂ covers at least a portion of the user's left side of the forehead. The third thermal camera and the fourth thermal camera are physically coupled to the frame, and located to the right and to the left of the symmetry axis, respectively. The third and fourth thermal cameras are each less than 10 cm away from the user's face and below the first and second thermal cameras.

The third thermal camera is configured to take thermal measurements of a third ROI (TH_{ROI3}), where ROI₃ covers at least a portion of the user's right upper lip. The fourth thermal camera is configured to take thermal measurements of a fourth ROI (TH_{ROI4}), where ROI₄ covers at least a portion of the user's left upper lip. Additionally, the third and fourth thermal cameras are located outside the exhale streams of the mouth and nostrils, and the thermal cameras are not in physical contact with their respective ROIs. Optionally, the first, second, third and fourth thermal cameras are located less than 3 cm away from the user's face. Optionally, the system includes a processor that is configured to utilize TH_{ROI1}, TH_{ROI2}, TH_{ROI3}, and TH_{ROI4} to a processor that is configured to identify the physiological response. In one example, the physiological reaction is indicative of an emotional state of the user, such as indicative of an extent to which the user felt at least one of the following emotions: anger, disgust, fear, joy, sadness, and surprise. In another example, the physiological reaction is indicative of an allergic reactions or a level of stress felt by the user.

In some embodiments, the Scheimpflug principle is utilized in order to achieve an extended depth of field (DOF). The Scheimpflug principle is a geometric rule that describes the orientation of the plane of focus of an optical system (such as a camera) when the lens plane is not parallel to the image plane. In one embodiment, a system comprises a frame configured to be worn on a user's head and a thermal camera, weighing below 10 g, which is physically coupled to the frame and located less than 5 cm away from the user's face. The thermal camera is configured to take thermal measurements of a region of interest (TH_{ROI}) on the user's face. In this embodiment, the thermal camera utilizes a Scheimpflug adjustment suitable for the expected position of the thermal camera relative to the ROI when the user wears the frame. When the lens and image planes are parallel, the depth of field (DoF) extends between parallel planes on either side of the plane of focus (PoF). When the Scheimpflug principle is employed, the DoF becomes wedge shaped with the apex of the wedge at the PoF rotation axis. The DoF is zero at the apex, remains shallow at the edge of the lens's field of view, and increases with distance from the camera. On a plane parallel to the image plane, the DoF is equally distributed above and below the PoF. This distribution can be helpful in determining the best position for the PoF. In one example, the Scheimpflug adjustment is achieved using at least one stepper motor, also known as step motor, which is a brushless DC electric motor that divides rotation into a number of steps. The motor's position can then be commanded to move and hold at one of these steps without any feedback sensor. In another example, the Scheimpflug adjustment is achieved using at least one brushed DC electric motor. In still another example, the Scheimpflug adjustment is achieved using at least one brushless DC motors. In yet another example, the Scheimpflug adjustment is achieved using at least one piezoelectric motor.

### BRIEF DESCRIPTION OF THE DRAWINGS

The embodiments are herein described by way of example only, with reference to the accompanying drawings. No attempt is made to show structural details of the embodiments in more detail than is necessary for a fundamental understanding of the embodiments. In the drawings:

FIG. 1a, FIG. 1b, FIG. 2a, and FIG. 2b illustrate various types of head mounted systems with cameras thereon, wherein the dotted circles and ellipses illustrate the region of interests of the cameras;

FIG. 3a and FIG. 3b illustrate various types of head mounted systems with cameras thereon, wherein the dotted lines illustrate the fields of view of the cameras;

FIG. 4a and FIG. 4b illustrate various potential locations to connect thermal cameras to various head mounted display frames in order to have at least some of the periorbital ROI within the field of view of one or more of the thermal cameras;

FIG. 5 illustrates the periorbital ROI;

FIG. 6, FIG. 7 and FIG. 8 illustrate various facial regions and related nomenclature; and

FIG. 9a and FIG. 9b are schematic illustration of computers able to realize one or more of the embodiments discussed herein.

### DETAILED DESCRIPTION

The term “thermal camera”, as used herein, refers to a non-contact device (i.e., not in physical contact with the measured area) based on a thermal sensor designed to measure wavelengths longer than 2500 nm. The thermal sensor may be used to measure spectral radiation characteristics of a black body at the user's body temperatures according to Planck's radiation law. Although the thermal camera may also measure wavelengths shorter than 2500 nm, a camera that measures near-IR (such as 700-1200 nm), and is not primarily designed for measuring wavelengths longer than 2500 nm, is referred to herein as near-IR camera and is not considered herein a thermal camera because it typically may not be used to effectively measure black body temperatures around 310 K. A thermal camera may include one or more sensing elements (that may also be referred to herein as sensing pixels or pixels). For example, a thermal camera may include just one sensing element (i.e., one sensing pixel, such as one thermopile sensor similar to Texas Instruments TMP006B Infrared Thermopile Sensor, or one pyroelectric sensor), or a focal-plane array containing multiple sensing elements (such as multiple thermopile sensing elements similar to Melexis MLX90621 16×4 thermopile array, or multiple microbolometer sensing elements similar to FLIR Lepton® 80×60 microbolometer sensor array).

When a thermal capturing device utilizes optics for its operation, then the term “thermal camera” may refer also to the optics (e.g., one or more lenses). When a thermal capturing device includes an optical limiter that limits the angle of view (such as in a pinhole camera, or a thermopile sensor inside a standard TO-5, TO-18, or TO-39 package with a window, or a thermopile sensor with a polished metal field limiter), then the term “thermal camera” may also refer to the optical limiter. “Optical limiter” may also be referred to herein as a “field limiter” or “field of view limiter”. Optionally, the field limiter may be made of a material with low emissivity and small thermal mass, such as Nickel-Silver and/or Aluminum foil. The term “thermal camera” may also cover a readout circuit adjacent to the thermal sensor, and/or the housing that holds the thermal sensor.

It is noted that the meaning of referring to the thermal camera as “not being in physical contact with the measured area” is that in a nominal operating condition there should be a space of at least 1 mm between the thermal camera (including its optics) and the user's skin. Furthermore, it is noted that sentences such as “the thermal camera is not in physical contact with the ROI” mean that the thermal camera utilizes a non-contact sensor that (i) is at a distance of at least 1 mm form the user's skin, and (ii) does not touch the ROI directly in a manner similar to a thermistor that requires physical contact with the ROI.

The term “thermal measurements of the ROI” (usually denoted TH_{ROI}) refers to at least one of temperature measurements and temperature change measurements. “Temperature measurements of the ROI” (usually denoted T_{ROI}) can be taken, for example, with a thermopile sensor or a microbolometer sensor, which measure the temperature at the ROI. “Temperature change measurements of the ROI” (usually denoted T_{ROI}) can be taken, for example, with a pyroelectric sensor that measures the temperature change at the ROI, or calculated by watching the changes in the temperature measurements taken at different times by a thermopile sensor or a microbolometer sensor. It is noted that the term microbolometer may refer to any type of bolometer sensor and its equivalents.

A more comprehensive discussion of thermal cameras, such as their various properties and configurations, is provided further below in this disclosure.

The term “circuit” is defined herein as an electronic device, which may be analog and/or digital, such as one or more of the following: an amplifier, a differential amplifier, a filter, analog and/or digital logic, a processor, a controller, a computer, an ASIC, and an FPGA.

As discussed above, collecting thermal measurements of various regions of a user's face can have many health-related (and other) applications. However, movements of the user and/or of the user's head can make acquiring this data difficult for many known approaches. Some embodiments described herein utilize various combinations of thermal cameras that are physically coupled to a frame of a head-mounted system (HMS), as the descriptions of the following embodiments show.

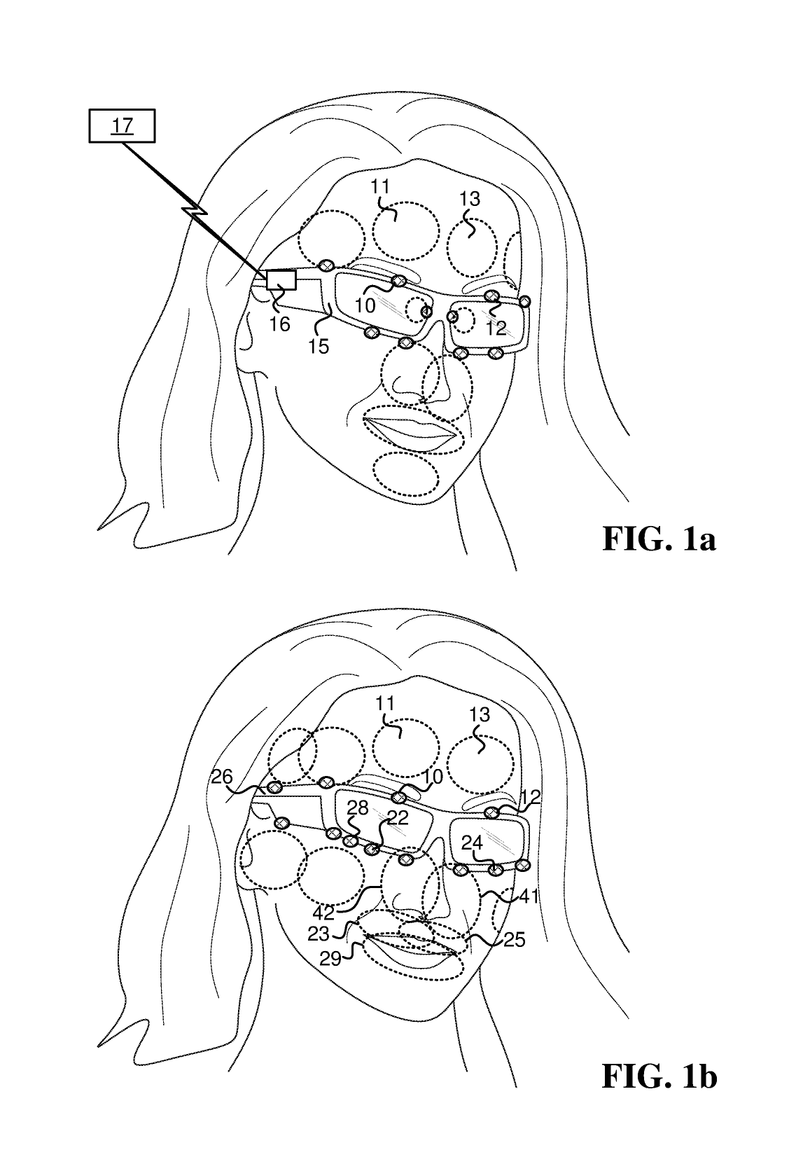

FIG. 1a illustrates one embodiment of a system that includes a first thermal camera 10 and a second thermal camera 12 that are physically coupled to a frame 15 configured to be worn on a user's head. The first thermal camera is configured to take thermal measurements of a first region of interest 11 (the “first region of interest” denoted ROI₁, and the “thermal measurements of ROI₁” denoted TH_{ROI1}), where ROI₁ 11 covers at least a portion of the right side of the user's forehead, and the second thermal camera is configured to take thermal measurements of a second ROI (TH_{ROI2}), wherein ROI₂ 13 covers at least a portion of the left side of the user's forehead.

In one embodiment, the system described above is configured to forward TH_{ROI1} and TH_{ROI2} to a processor 16 configured to identify a physiological response based on TH_{ROI1} and TH_{ROI2}. The processor 16 may be located on the user's face, may be worn by the user, and/or may be located in a distance from the user, such as on a smartphone, a personal computer, a server, and/or on a cloud computer. The wearable processor 16 may communicate with the non-wearable processor 17 using any appropriate communication techniques.

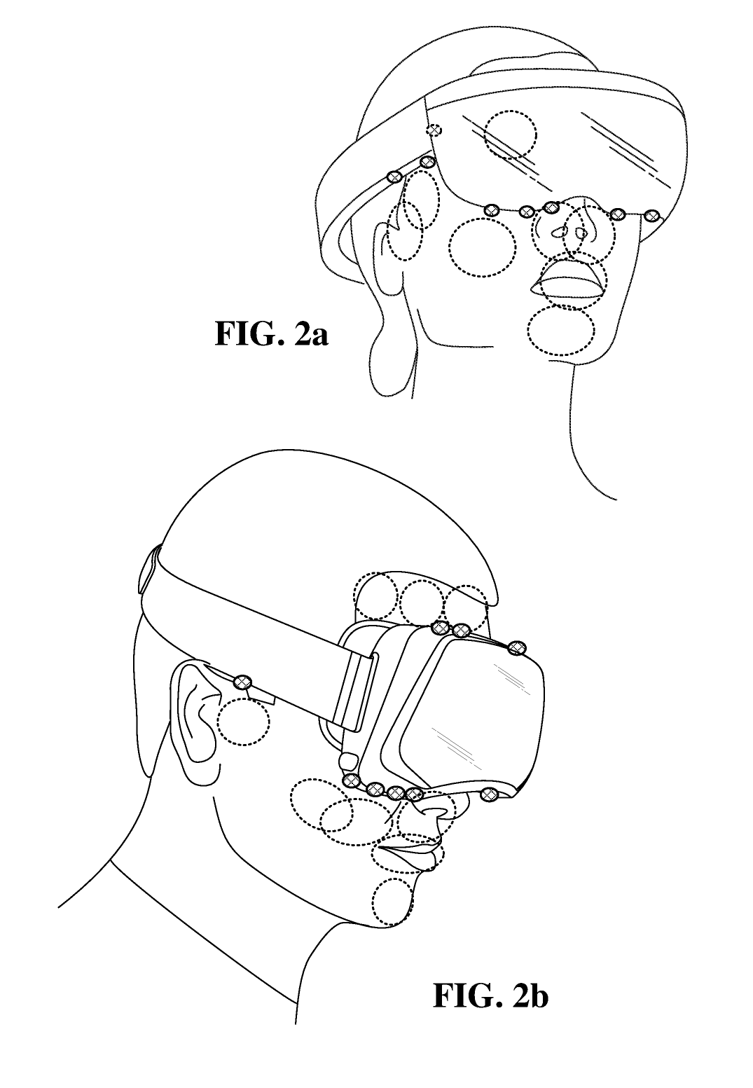

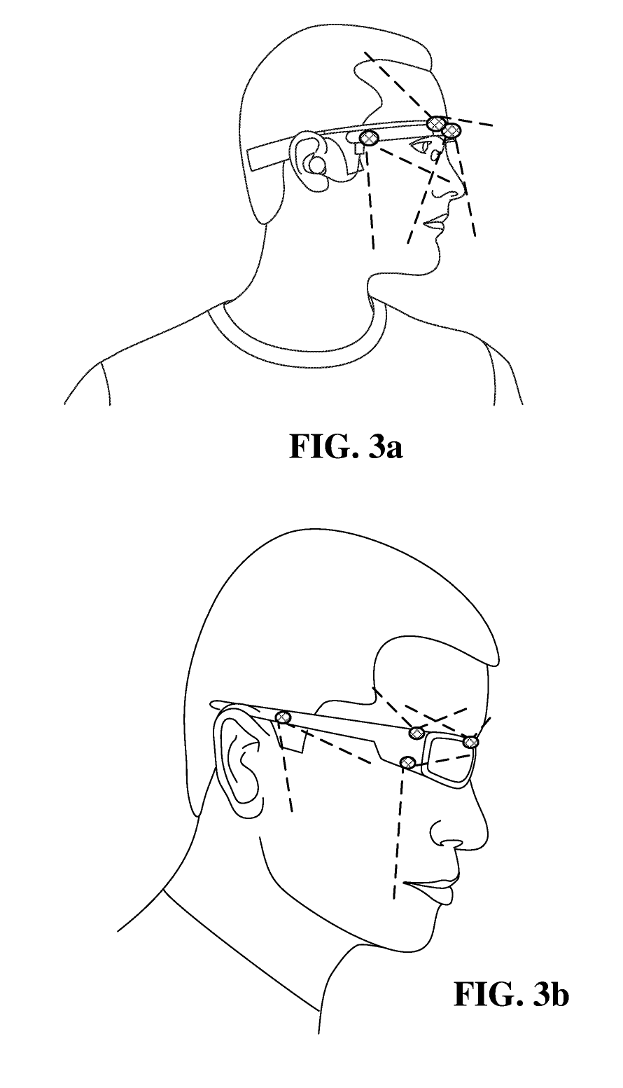

FIG. 1b, FIG. 2a, and FIG. 2b illustrate various types of head-mounted systems with cameras thereon; the dotted circles and ellipses illustrate the ROIs of the cameras. The cameras may be thermal cameras and/or visible light cameras. In the illustrations, cameras are designated by a button like symbol (see for example thermal camera 10 in FIG. 1a). FIG. 3a and FIG. 3b illustrate a side view of various types of head mounted systems with cameras thereon; the dotted lines illustrate the Fields Of View (FOVs) of the cameras. The cameras may be thermal cameras and/or visible light cameras.

It is to be noted that the positions of the cameras in the figures are just for illustration. The cameras may be placed at other positions on the HMS. One or more of the visible light cameras may be configured to capture images at various resolutions or at different frame rates. Many video cameras with a small form-factor, such as those used in cell phones or webcams, for example, may be incorporated into some of the embodiments.

Furthermore, illustrations and discussions of a camera represent one or more cameras, where each camera may be configured to capture the same field of view (FOV), and/or to capture different FOVs (i.e., they may have essentially the same or different FOVs). Consequently, each camera may be configured to take measurements of the same regions of interest (ROI) or different ROIs on a user's face. In some embodiments, the one or more of the cameras may include one or more elements, such as a gyroscope, an accelerometer, and/or a proximity sensor. Optionally, other sensing devices may be included within a camera, and/or in addition to the camera, and other sensing functions may be performed by one or more of the cameras.

In some embodiments, because facial structures may differ from user to user, the HMS may calibrate the direction, position, algorithms, and/or characteristics of one or more of the cameras and/or light sources based on the facial structure of the user. In one example, the HMS calibrates the positioning of a camera in relation to a certain feature on the user's face. In another example, the HMS changes, mechanically and/or optically, the positioning of a camera in relation to the frame in order to adapt itself to a certain facial structure.

It is noted that an object is not in the FOV of a camera when it is not located in the angle of view of the camera and/or when there is no line of sight from the camera to the object, where “line of sight” is interpreted in the context of the spectral bandwidth of the camera.

It is further noted that phrases of the form of “the angle between the optical axis of a camera and the Frankfort horizontal plane is greater than 20°” refer to absolute values (which may take +20° or −20° in this example) and are not limited to just positive or negative angles, unless specifically indicated such as in a phrase having the form of “the optical axis of the camera points at least 20° below the Frankfort horizontal plane” where it is clearly indicated that the camera is pointed downwards.

In one example, “a frame configured to be worn on the user's head” is interpreted as a frame that loads more than 50% of its weight on the user's head. For example, the frame in Oculus Rift and HTC Vive includes the foam placed on the user's face and the straps; the frame in Microsoft HoloLens includes the adjustment wheel in the headband placed on the user's head. In another example, “a frame configured to be worn on the user's head” may be similar to an eyeglasses frame, which holds prescription and/or UV-protective lenses.

Some of the various systems described in this disclosure, e.g., as illustrated in FIG. 1a to FIG. 3b, may involve at least two thermal cameras that are used to take thermal measurements of possibly different ROIs. An example of such as system in described in the embodiment below includes at least a frame, a first thermal camera, and a second thermal camera.

The frame is configured to be worn on a user's head. Optionally, the frame may be any of the frames of HMSs described herein, such as a frame of glasses or part of a head-mounted display (e.g., an augmented reality system, a virtual reality system, or a mixed reality system).

The first thermal camera is physically coupled to the right side of the frame and is located less than 10 cm away from the user's face. Herein, “cm” refers to centimeters. The first thermal camera is configured to take thermal measurements of a first region of interest (TH_{ROI1}). Optionally, ROI₁ covers at least a portion of the right side of the user's forehead, and the system does not occlude ROI₁. In one example, the first thermal camera may be thermal camera 10 in FIG. 1a.

It is noted that the distance in sentences such as “a thermal camera located less than 10 cm away from the user's face” refers to the shortest possible distance between the thermal camera and the face. For example, the shortest distance between sensor 10 and the user's face in FIG. 1a is from sensor 10 to the lower part of the right eyebrow, and not from sensor 10 to ROI 11.

The second thermal camera is physically coupled to the left side of the frame and is located less than 10 cm away from the user's face. The second thermal camera is configured to take thermal measurements of a second region of interest (TH_{ROI2}). Optionally, ROI₂ covers at least a portion of the right side of the user's forehead, and the system does not occlude ROI₂. In one example, the second thermal camera may be thermal camera 12 in FIG. 1a.

It is to be noted that because the thermal cameras are coupled to the frame, in some embodiments, challenges such as dealing with complications caused by movements of the user, ROI alignment, tracking based on hot spots or markers, and motion compensation in the IR video—are simplified, and may be even eliminated.

In one embodiment, the system described above (e.g., the frame or other elements belonging to an HMS) does not occlude ROI₁ and ROI₂, and the overlap between ROI₁ and ROI₂ is less than 80% of the smallest area from among the areas of ROI₁ and ROI₂. Additionally, both the first and second thermal cameras are lightweight, weighing less than 5 g each (herein “g” denotes grams).

In is to be noted that sentences in the form of “the system/camera does not occlude the ROI” are defined herein as follows. The ROI is not considered occluded when more than 80% of the ROI can be observed by a third person standing in front of the user and looking at the user's face; while the ROI is considered occluded when more than 20% of the ROI cannot be observed by the third person.

In one embodiment, at least one of the first and second thermal cameras weighs below 1 g. Additionally or alternatively, at least one of the first and second thermal cameras may be based on at least one of the following uncooled sensors: a thermopile, a pyroelectric, and a microbolometer.

In one embodiment, the first and second thermal cameras are not in physical contact with their corresponding ROIs. Additionally, the thermal cameras remain pointed at their corresponding ROIs when the user's head makes angular movements as a result of being coupled to the frame. In one example, angular movements are interpreted as movements of more than 45°. In another example, the locations of the first and second cameras relative to the user's head do not change even when the user's head performs wide angular and lateral movements, where wide angular and lateral movements are interpreted as angular movements of more than 60° and lateral movements of more than 1 meter.

Thermal measurements taken with the first and second thermal cameras may have different properties, in different embodiments. In particular, the measurements may exhibit certain measurement errors for the temperature, but when processed, may result is lower errors for the change of temperature (ΔT) as discussed below.

In one example, the first and second thermal cameras measure temperature with a possible measurement error above ±1.0° C. and provide temperature change (ΔT) with an error below ±0.10° C. Optionally, the system includes a processor configured to estimate a physiological response based on ΔT measured by the first and second thermal cameras.

In another example, the first and second thermal cameras measure temperature with a possible measurement error above ±0.20° C. and provide temperature change (ΔT) with an error of below ±0.050° C. Optionally, the system includes a processor configured to estimate a physiological response based on ΔT measured by the first and second thermal cameras.

In yet another example, the first and second thermal cameras measure temperatures at ROI₁ and ROI₂, and the system's nominal measurement error of the temperature at ROI₁ and ROI₂ (ERR_{TROI}) is at least five times the system's nominal measurement error of the temperature changes at the ROI₁ and ROI₂ (ERR_{ΔTROI}) when the user's head makes angular movements also above 0.1 rad/sec (radians per second). Optionally, the system includes a processor configured to identify affective response that causes a temperature change at ROI₁ and ROI₂ that is below ERR_{TROI} and until ERR_{ΔTROI}.

Measurements of the thermal cameras may be utilized for various calculations in different embodiments. In one example, the first and second thermal cameras measure temperatures at ROI₁ and ROI₂, respectively. The system, this embodiment, may include a circuit that is configured to: receive a series of temperature measurements at ROI₁ and calculate temperature changes at ROI₁ (ΔT_{ROI1}), receive a series of temperature measurements at ROI₂ and calculate temperature changes at ROI₂ (ΔT_{ROI2}), and utilize ΔT_{ROI1} and ΔT_{ROI2} to identify a physiological response. Optionally, the system's nominal measurement error of the temperatures at ROI₁ is at least twice the system's nominal measurement error of the temperature changes at ROI₁ when the user's head makes angular movements also above 0.1 rad/sec. Optionally, the system's nominal measurement error of the temperatures at ROI₁ is at least five times the system's nominal measurement error of the temperature changes at ROI₁ when the user's head makes angular movements also above 0.5 rad/sec.

In different embodiments, the ROIs mentioned above may cover slightly different regions on the user's face. In one example, the right side of the user's forehead covers at least 30% of ROI₁, and the left side of the user's forehead covers at least 30% of ROI₂. In another example, the right side of the user's forehead covers at least 80% of ROI₁, and the left side of the user's forehead covers at least 80% of ROI₂.

In some embodiments, the system described above is configured to forward TH_{ROI1} and TH_{ROI2} to a processor configured to identify a physiological response based on TH_{ROI1} and TH_{ROI2}. Optionally, the physiological response is indicative of at least one of the following: stress, mental workload, fear, sexual arousal, anxiety, pain, pulse, headache, and stroke. Optionally, the physiological response is indicative of stress level, and further the system further includes a user interface configured to alert the user when the stress level reaches a predetermined threshold. Optionally, TH_{ROI1} and TH_{ROI2} are correlated with blood flow in the frontal vessel of the user's forehead, which may be indicative of mental stress.

A specific signal that may be identified, in some embodiments, involves the blood flow in the user's body. For example, in one embodiment, ROI₁ covers at least a portion of the right side of the frontal superficial temporal artery of the user, and ROI₂ covers at least a portion of the left side of the frontal superficial temporal artery of the user. Optionally, the system in this embodiment is configured to forward TH_{ROI1} and TH_{ROI2} to a processor that is configured to identify, based on TH_{ROI1} and TH_{ROI2}, at least one of the following: arterial pulse, headache, and stroke.

The following is an example of how some embodiments described herein may be utilized to obtain values of a physiological signal that has periodic features, such as pulse or respiration. Optionally, in these embodiments, the thermal camera(s) may include multiple sensing elements, and a computer may extract temporal signals for individual pixels inside ROI₁ and/or ROI₂, and/or extract temporal signals for pixel clusters inside ROI₁ and/or ROI₂, depending on the movement and the noise level. The calculation of the physiological signal may include harmonic analysis, such as a fast Fourier transform, applied to the temperature signal and/or temperature change signal of each pixel, or pixel clusters, over time in a sliding window, which may be followed by a non-linear filter to reduce low-frequency signal leakage in the measured frequency range. In cases where some pixels may be less informative than others, a clustering procedure may be implemented to remove the outliers. Following that, the frequency peaks in the set of pixels of interest may be used to vote for the dominant frequency component, the bin with the most votes is selected as the dominant frequency, and the estimate of the physiological signal may be obtained from the median filtered results of the dominant frequency components in a small sliding window.

One example of a contact-free heart rate and respiratory rate detection through measuring changes to infrared light emitted near the superficial blood vessels or the nasal area, respectively, is described in the reference Yang, M., Liu, Q., Turner, T., & Wu, Y. (2008), “Vital sign estimation from passive thermal video”, In Computer Vision and Pattern Recognition, 2008 (pp. 1-8), CVPR 2008 IEEE. Pulsating blood flow induces subtle periodic temperature changes to the skin above the superficial vessels by heat diffusion, which may be detected by thermal video to reveal the associated heart rate. The temperature modulations may be detected through pixel intensity changes in the ROI using a thermal camera, and the corresponding heart rate may be measured quantitatively by harmonic analysis of these changes on the skin area above the superficial temporal artery (in this context, “the skin area above the artery” refers to “the skin area on top of the artery”).

The temperature modulation level due to blood pulsating is far less than normal skin temperature, therefore, in one embodiment, the subtle periodic changes in temperature are quantified based on differences between image frames. For example, after an optional alignment, the frame differences against a certain reference frame are calculated for every frame, based on corresponding pixels or corresponding pixel clusters. The temperature differences may look like random noise in the first several frames, but a definite pattern appears close to half of the pulse period; then the temperature differences become noisy again as approaching the pulse period. The heart rate is estimated by harmonic analysis of the skin temperature modulation above the superficial temporal artery. In one embodiment, a similar method is applied for respiration rate estimation by measuring the periodic temperature changes around the nasal area.

In one embodiment, ROI₁ covers at least a portion of the right side of the superficial temporal artery of the user, and ROI₂ covers at least a portion of the left side of the superficial temporal artery of the user. Optionally, in this embodiment, the system is configured to forward TH_{ROI1} and TH_{ROI2} to a processor configured to identify, based on TH_{ROI1} and TH_{ROI2}, at least one of the following: arterial pulse, headache, and stroke. FIG. 7 in U.S. Pat. No. 8,360,986 awarded to Farag et al illustrates the right and left superficial temporal artery ROIs of one person. The locations and dimensions of the right and left superficial temporal artery ROIs may change to some extent between different people. Due to the inherent benefits obtained from the disclosed head mounted thermal cameras, it may be enough that ROI₁ and ROI₂ cover just a portion of the right and left superficial temporal artery ROIs. Additionally or alternatively, ROI₁ and ROI₂ may cover greater areas than the ROIs illustrated in FIG. 7 in U.S. Pat. No. 8,360,986.

Another example of a system that includes thermal cameras that take measurements of certain regions of a user's face is given is the following description. In one embodiment, a wearable system configured to take thermal measurements that enable identification of a physiological response includes at least a frame and first, second, third, and fourth thermal cameras. The frame is configured to be worn on a user's head, and the first, second, third and fourth thermal cameras remain pointed at their respective ROIs when the user's head makes angular movements. For example, the first, second, third and fourth thermal cameras may remain pointed at their respective ROIs when the user's head makes angular movements that exceed 0.1 rad/sec. An illustration of an example of such a system is given in FIG. 1b.

The first and second thermal cameras are physically coupled to the frame and are located to the right and to the left of the symmetry axis that divides the user's face to the right and left sides, respectively. Additionally, each of these thermal cameras is less than 10 cm away from the user's face. The first thermal camera 10 is configured to take thermal measurements of a first region of interest (TH_{ROI1}), where ROI₁ 11 covers at least a portion of the right side of the user's forehead. The second thermal camera 12 is configured to take thermal measurements of a second region of interest (TH_{ROI2}), where ROI₂ 13 covers at least a portion of the user's left side of the forehead. The third thermal camera 22 and the fourth thermal camera 24 are physically coupled to the frame 26, and located to the right and to the left of the symmetry axis, respectively. The third and fourth thermal cameras are each less than 10 cm away from the user's face and below the first and second thermal cameras. The third thermal camera 22 is configured to take thermal measurements of a third ROI (TH_{ROI1}), where ROI₃ 23 covers at least a portion of the user's right upper lip. The fourth thermal camera 24 is configured to take thermal measurements of a fourth ROI (TH_{ROI4}), where ROI₄ 25 covers at least a portion of the user's left upper lip. Additionally, the third and fourth thermal cameras are located outside the exhale streams of the mouth and nostrils, and the thermal cameras are not in physical contact with their respective ROIs. Optionally, the first, second, third and fourth thermal cameras are located less than 3 cm away from the user's face.

In one embodiment, the system described above is configured to forward TH_{ROI1}, TH_{ROI2}, TH_{ROI1}, and TH_{ROI4} to a processor that is configured to identify the physiological response. In one example, the physiological response is indicative of an emotional state of the user, such as indicative of an extent to which the user felt at least one of the following emotions: anger, disgust, fear, joy, sadness, and surprise. In another example, the physiological response is indicative of a level of stress felt by the user. In yet another example, the physiological response is indicative of an allergic reaction of the user. And in still another example, the physiological response is indicative of a level of pain felt by the user.

In one embodiment, the overlap between ROI₁ and ROI₂ is lower than 50% of the smallest area from among the areas of ROI₁ and ROI₂, and the overlap between ROI₃ and ROI₄ is lower than 50% of the smallest area from among the areas of ROI₃ and ROI₄. In another embodiment, there is no overlap between ROI₁ and ROI₂, and there is no overlap between ROI₃ and ROI₄.

In one embodiment, the system described above may include an additional fifth camera. In one example, the fifth thermal camera coupled to the frame, pointed at a fifth ROI (ROI₅) that covers at least a portion of the user's nose. In another example, the fifth thermal camera coupled to the frame, pointed at a fifth ROI (ROI₅) that covers at least a portion of periorbital region of the user's face.

In addition to thermal cameras, in some embodiments, one or more visible light cameras may be utilized in order to acquire measurements that may be utilized for various applications. Herein, the term “visible light camera” refers to a camera designed to detect at least some of the visible spectrum. Examples of visible light sensors include active pixel sensors in complementary metal-oxide-semiconductor (CMOS), and semiconductor charge-coupled devices (CCD). The following is an example of such a system.

In one embodiment, a system configured to take thermal measurements and visible light measurements of a user's face from fixed relative positions includes at least a frame, a first a thermal camera, a second thermal camera, and a visible light camera. In this embodiment, the visible light camera and the first and second thermal cameras each weighs less than 5 grams. The frame is configured to be worn on the user's head, and the first thermal camera, the second thermal camera, and the visible light camera are physically coupled to the frame.

The first thermal camera is configured to take thermal measurements of a first region of interest (TH_{ROI1}), where ROI₁ covers at least part of the area around the user's eyes. The second thermal camera is configured to take thermal measurements of a second ROI (TH_{ROI2}), where ROI₂ covers at least part of the user's upper lip and the system does not occlude ROI₂. The visible light camera is configured to take images of a third ROI (IMROI₃), where ROI₃ covers at least part of ROI₂. The thermal cameras and the visible light camera maintain fixed positioning relative to each other and relative to their corresponding ROIs when the user's head makes angular movements also above 0.1 rad/sec.

In one embodiment, the system described above optionally includes a processor that is configured to train a machine learning-based model for the user based on TH_{ROI1} and IROI2. Optionally, the model identifies affective response of the user.

In one embodiment, the visible light camera comprises a lens that is tilted according to Scheimpflug principle in order to achieve an extended depth of field (DOF) that provides a sharper image of ROI₂ compared to the image of ROI₂ that would have been obtained from the same visible light camera using a non-tilted lens.

In another embodiment, the second thermal camera comprises a focal-plane array (FPA) and a lens that is tilted according to Scheimpflug principle in order to achieve an extended depth of field (DOF) that provides a sharper image of ROI₁ compared to the image of ROI₁ that would have been obtained from the same thermal camera using a non-tilted lens.

The following is a more detailed discussion about utilization of the Scheimpflug principle in order to achieve an extended depth of field (DOF). In one embodiment, a system comprises a frame configured to be worn on a user's head and a thermal camera, weighing below 10 g, which is physically coupled to the frame and located less than 5 cm away from the user's face. The thermal camera is configured to take thermal measurements of a region of interest (TH_{ROI}, ROI) on the user's face. In this embodiment, the thermal camera utilizes a Scheimpflug adjustment suitable for the expected position of the thermal camera relative to the ROI when the user wears the frame.

The Scheimpflug principle is a geometric rule that describes the orientation of the plane of focus of an optical system (such as a camera) when the lens plane is not parallel to the image plane. Herein “Scheimpflug adjustment” refers to orientation greater than 2°, which is not due to a manufacturing error.

When the lens and image planes are parallel, the depth of field (DoF) extends between parallel planes on either side of the plane of focus (PoF). When the Scheimpflug principle is employed, the DoF becomes wedge shaped with the apex of the wedge at the PoF rotation axis. The DoF is zero at the apex, remains shallow at the edge of the lens's field of view, and increases with distance from the camera. On a plane parallel to the image plane, the DoF is equally distributed above and below the PoF. This distribution can be helpful in determining the best position for the PoF.

Some example of references that may be relevant to some of the embodiments related to Scheimpflug principle include the following: Depth of field for the tilted lens plane, by Leonard Evens, 2008; Tilt and Shift Lenses, by Lester Wareham (http://www.zen20934.zen.co.uk/photography/tiltshift.htm); Addendum to focusing the view camera, by Harold M. Merklinger, World Wide Web Edition, 1993; U.S. Pat. No. 6,963,074; US Patent Application 20070267584; and US Patent Application 20070057164.

In one example, the Scheimpflug adjustment is achieved using at least one stepper motor, also known as step motor, which is a brushless DC electric motor that divides rotation into a number of steps. The motor's position can then be commanded to move and hold at one of these steps without any feedback sensor. In another example, the Scheimpflug adjustment is achieved using at least one brushed DC electric motor. In still another example, the Scheimpflug adjustment is achieved using at least one brushless DC motors. In yet another example, the Scheimpflug adjustment is achieved using at least one piezoelectric motor, as such described in the reference Morita, T. (2003), “Miniature piezoelectric motors”, Sensors and Actuators A: Physical, 103(3), 291-300. And in still another example, the Scheimpflug adjustment is achieved using at least one micro-motion motor, such as described in the reference Ouyang, P. R., Tjiptoprodjo, R. C., Zhang, W. J., & Yang, G. S. (2008), “Micro-motion devices technology: The state of arts review”, The International Journal of Advanced Manufacturing Technology, 38(5-6), 463-478.

The Scheimpflug principle may be utilized, in some embodiments, to reduce computational power required to generate a focused image of the face. For example, a system may include a frame configured to be worn on a user's head and a camera (visible light or thermal), weighing below 10 g, which is physically coupled to the frame and located less than 5 cm away from the user's face. In this example, the camera is configured to capture an ROI on the user's face. The camera is coupled to the frame and is positioned in an acute angle relative to the ROI. For example, the acute angle may be less than 20, 30, 40, 50, 60, or 90 degrees.

The system described above further includes a Scheimpflug principle camera coupled to the frame in an acute angle relative to the ROI and a controller that is configured to rotate at least one of the optics and sensor according to Scheimpflug principle to achieve a focused image of the ROI.

The Scheimpflug principle may be utilized to operate a light field camera comprising a Scheimpflug adjustment mechanism. In one embodiment, a system includes at least a frame configured to be worn on a user's head and a light field camera, weighing below 10 g, which is physically coupled to the frame and located less than 5 cm away from the user's face. The camera is configured to capture an ROI on the user's face. Additionally, the camera is coupled to the frame in an acute angle relative to the ROI. Operating the light field camera may involve the following steps:

In Step 1, autofocusing Scheimpflug adjustment mechanism of the light field camera by changing the relative angle between a sensor and an objective lens. Optionally, the autofocusing of the Scheimpflug adjustment mechanism operates based on the principle that scene points that are not in focus are blurred while scene points in focus are sharp. In one example, this step involves studying a small region around a given pixel; this region will be sharper in the image as the Scheimpflug correction is better, and it will get more and more blurred as the Scheimpflug correction does not fit. In another example, this step involves using the variance of the neighborhood around each pixel as a measure of sharpness, where the Scheimpflug correction is better as the variance of its neighborhood is maximum.

In Step 2, capturing an image, while implementing a predetermined blurring, at a certain Scheimpflug angle.

And in Step 3, decoding the predetermined blurring as function of the certain Scheimpflug angle. In one example, a focused sensor measures a spectral slice that tilts when out-of-focus. After applying the Scheimpflug correction, the spectral slice would tilt differently, and the decoding the predetermined blurring as function takes that into account when decoding the blurred image.

There are various ways in which the Scheimpflug adjustment mechanism may be implemented. In one example, the Scheimpflug adjustment mechanism comprises a mirror that changes its angle. In another example, the Scheimpflug adjustment mechanism comprises a device that changes the angle of the objective lens (not the blurring element, such as the micro-lenses or the mask) relative to the sensor. And in still another example, the Scheimpflug adjustment mechanism comprises a device that changes the angle of the sensor relative to the objective lens.

In another embodiment, method for operating a light field camera comprising a Scheimpflug adjustment mechanism, involves performing the following steps utilizing the system described above:

In Step 1, autofocusing a Scheimpflug adjustment mechanism comprised in the camera by changing the relative angle between a blurring element and a sensor.

In Step 2, capturing an image, while implementing a predetermined blurring, at a certain Scheimpflug angle.

And in Step 3, decoding the predetermined blurring as function of the certain Scheimpflug angle between the blurring element and the sensor.

In one embodiment, a method for selecting a Scheimpflug adjustment angle based on a depth map, which is utilized by the system described above, includes at least the following steps:

In Step 1, capturing a picture using the light field camera.

In Step 2, extracting a depth map from the picture.

In Step 3, utilizing the depth map to find the Scheimpflug adjustment angle that maximize the image sharpness.

And In Step 4, sending a command to apply a Scheimpflug adjustment angle according to the Scheimpflug adjustment angle that maximize the image sharpness.

In one embodiment, the motors are essentially continuous and the applied Scheimpflug adjustment angle is essentially the Scheimpflug adjustment angle that maximize the image sharpness. In another embodiment, the motors are stepper and the applied Scheimpflug adjustment angle is the closest angle to the Scheimpflug adjustment angle that maximize the image sharpness.

Following are embodiments of various applications for which the systems described above (e.g., systems corresponding to FIG. 1a to FIG. 3b) may be utilized. The applications may involve detection of various physiological reactions, such as detecting an allergic reaction, stress, or various security-related applications.

One application for which thermal measurements of one or more ROIs on the face may be useful is do detect an onset and/or extent of an allergic reaction. In one embodiment, a system configured to determine an extent of an allergic reaction of a user includes at least a frame, a thermal camera, and a circuit.

The frame is configured to be worn on the user's head and the thermal camera is physically coupled to the frame and located less than 10 cm away from the user's face. The thermal camera, which weighs less than 5 gram, is configured to take thermal measurements of at least part of the user's nose (TH_{N}). Optionally, the thermal camera is based on at least one of the following uncooled sensors: a thermopile, a pyroelectric, and a microbolometer. Optionally, the thermal camera is not in physical contact with the nose, and remains pointed at the nose when the user's head makes angular movements also above 0.1 rad/sec. Optionally, the thermal camera is located less than 3 cm away from the user's face and weighs below 1 g. Optionally, the system does not occlude the ROI. Additional discussion regarding some of the properties of the thermal camera (e.g., accuracy) is given further below.

The circuit is configured to determine an extent of the allergic reaction based on TH_{N}. Optionally, determining the extent of the allergic reaction involves determining whether there is an onset of an allergic reaction. Optionally, determining the extent of the allergic reaction involves determining a value indicative of the severity of the allergic reaction. Optionally, the measurements taken by the thermal camera, which are utilized by the circuit to determine the extent of the allergic reaction, may include measurements of regions near the user's mouth (e.g., the lips and/or edges of the mouth).

It is to be noted that while the description above describes a single thermal camera, in some embodiments, multiple thermal cameras may be utilized to obtain measurements from various ROIs such as different regions/sides of the user's nose and/or different regions/sides of the user's mouth. Some examples of possible locations for one or more thermal cameras coupled to the frame and their corresponding ROIs are given in FIG. 1a and FIG. 1b. For example, temperature measurements at ROIs 41, 42, 23, 25, and/or 29 may be utilized, in some embodiments, for the detection of an onset of an allergic reaction and/or determination of the extent of the allergic reaction.

In some embodiments, the measurements TH_{N} are represented as time series data, which includes values indicative of the temperature (or change to temperature) at an ROI that includes part of the user's nose at different times. In different embodiments, these measurements may be taken at different intervals, such as a few times a second, once a second, every few seconds, once a minute, and in some cases, every few minutes.

In some embodiments, the allergic reaction may involve one or more of the following reactions of the immune system: allergic rhinitis, atopic dermatitis, and anaphylaxis. Optionally, one of the manifestations of the allergic reaction may be a rise in the temperature at various regions of the face, such as the nose and/or the mouth.

In some embodiments, the allergic reaction may be in response to various types of allergens such as inhaled allergens, food, drugs, and/or various chemicals which the user may come in contact with (e.g., via the skin). In some embodiments, the allergic reaction is a response to one or more of the following allergens: pollen, dust, latex, perfume, a drug, peanuts, eggs, wheat, milk, and seafood.

Herein, an “onset of an allergic reaction” refers to an allergic reaction that is happening, i.e., at least some of activity of the immune system related to the allergic reaction is taking place and/or various symptoms of the allergic reaction are beginning to manifest. The activity and/or symptoms may continue to occur even beyond a point in time identified as corresponding to an onset of the allergic reaction. Additionally, in some cases, at the time an onset of an allergic reaction is identified, a user having the allergic reaction may not be aware of the allergic reaction, e.g., because the symptoms are not strong enough at the time. Thus, being notified about an onset of an allergic reaction before its full manifestation may have an advantage, in some embodiments, of allowing the user to take early action to alleviate and/or decrease the symptoms (e.g., take antihistamines), which may help to reduce to overall effects of the allergic reaction on the user.

In one embodiment, the ROI, of which measurements of thermal camera are taken, is the nasal area, and the circuit is further configured to detect an early rise in nasal temperature, which may be evident before the user is aware of the symptoms of the allergy reaction, and alert the user of possible allergy reaction. The reference Clark, A. T., Mangat, J. S., Tay, S. S., King, Y., Monk, C. J., White, P. A., & Ewan, P. W. (2007), “Facial thermography is a sensitive and specific method for assessing food challenge outcome”, Allergy, 62(7), 744-749, shows the fast increase in mean nasal temperature. For example, a fast increase due to an allergic reaction may correspond to an increase of more than 0.8° C. within a period of less than 30 minutes, 20 minutes, or even a shorter period than that (herein ° C. refers to Celsius degrees). Additionally, the reference Clark, A., Mangat, J., King, Y., Islam, S., Anagnostou, K., Foley, L., & Ewan, P. (2012), “Thermographic imaging during nasal peanut challenge may be useful in the diagnosis of peanut allergy”, Allergy, 67(4), 574-576, illustrates the fast response to nasal challenge, which can be used as a rapid, safe and objective clinical allergy test together with the head mounted thermal camera.

In one embodiment, upon identifying such an increase in temperature, the system can identify the potential cause to be one of the items to which the user was exposed during the preceding 20 minutes, or even during the preceding 10 minutes, or even during the preceding than the last 5 minutes.

The circuit may be any of the various types of circuits mentioned in this disclosure, e.g., it may be a processor, an ASIC, or an FPGA. In one example, the circuit is the circuit 16 described in FIG. 1a. In some embodiments, the circuit may be coupled to the frame and/or to an HMS of which the frame is a part. In other embodiments, the circuit may belong to a device carried by the user (e.g., a processor of a smartwatch or a smartphone).

In some embodiments, determining the extent of the allergic reaction is done by a circuit that is remote from the user. For example, the circuit may belong to cloud-based server, which receives TH_{N}, processes those values, and returns a result to the user (e.g., an alert regarding an onset of an allergic reaction).

Determining whether the user is experiencing an onset of an allergic reaction may be done by examining various properties of TH_{N}. For example, an onset may be detected if the rise in the temperature of an ROI in the nasal area and/or the mouth exceeds a certain threshold value such as, 0.5° C., 0.8° C., 1.0° C., or some other value greater than 0.5° C. and lower than 2.0° C. Optionally, the onset is detected if the rise exceeding the certain value occurs within a short period of time, such as 2 minutes, 5 minutes, 10 minutes, 15 minutes, 20 minutes, 25 minutes, 30 minutes, or some other period of time greater than 2 minutes and lesser than two hours.

In a similar fashion, determining the extent of an allergic reaction may also be done by examining various properties of TH_{N}. In one example, a value representing the extent of the allergic reaction is dependent on the value of the maximum increase detected in the temperature of a relevant ROI (e.g., nasal area and/or the mouth), such that the higher the temperature change, the greater the extent of the allergic reaction. In another example, a value representing the extent of the allergic reaction is dependent on the value representing the speed in which the increase detected in the temperature of a relevant ROI (e.g., nasal area and/or the mouth) reached a certain threshold (e.g., 0.5° C., 0.8° C., or 1.0° C.), such that the faster the certain threshold is reached, the greater the extent of the allergic reaction. In still another example, a value representing the extent of the allergic reaction is dependent is dependent on the area under a curve representing the change in the temperature of a relevant ROI (e.g., nasal area and/or the mouth) over time, such that the larger the area under the curve, the greater the extent of the allergic reaction.

When determining the extent of the allergic reaction, in some embodiments, additional inputs other than TH_{N} may be utilized. In one example, measurements of the environment taken with sensors may be utilized for this purpose. For example, the measurements may correspond to environmental parameters such as temperature, humidity, UV radiation levels, etc. In another example, the additional inputs may comprise values indicative of activity of the user, such as inputs from movement sensors and/or accelerometers. In still another example, the additional inputs may comprise temperature values of the user's body and/or cutaneous temperatures of other regions of the user's face and/or body (e.g., regions other than the nasal and/or mouth areas).

The various inputs described above may be utilized, in some embodiments, by the circuit to make more accurate determinations regarding the allergic reaction. For example, these inputs may be utilized in order to rule out false positives in which the ROIs may display an increase in temperature than is not due to an allergic reaction, such as temperature increases due to the environment (e.g., when exposed to the sun) and/or temperature increases due to the user's activity (e.g., while running or exercising). Additionally or alternatively, measurements of temperature from other regions may serve to normalize the values measured at the ROI. For example, if there is a change to the temperature at the forehead that is similar to the change in the nasal area, then in some cases, this may indicate that the user is not having an allergic reaction (even if the change is significant, such as exceeding 1.0° C.).

In some embodiments, determining, based on TH_{N}, the extent of the allergic reaction, may be done utilizing a machine learning-based model. In such embodiments, the circuit may compute various features derived from TH_{N} e.g., values of the temperature or change in temperature at different preceding times, and/or the change in temperature relative to various preceding points in time), and utilize the model to generate an output indicative of the extent of the allergic reaction. Optionally, features may include values derived from one or more of the additional input sources described above (e.g., environmental measurements, user activity signals, and/or temperature measured at other reasons).

In some embodiments, the model is generating based on labeled training data that includes samples including samples that each include feature values derived from values of TH_{N} and labels indicative of whether there is an allergic reaction (e.g., a label indicating whether there is an onset and/or a value indicative of the severity of the allergic reaction). Optionally, some labels may be provided by the user to samples generated from measurements of the user (thus, the model may be considered a personalized model of the user).

It is to be noted that an extent of an allergic reaction may be expressed using various values. In one example, the extent is treated as a binary value (e.g., allergic reaction vs. no allergic reaction). In another example, the extent is a categorical value indicative of the severity of the reaction (e.g., no reaction, low-level allergic reaction, medium allergic reaction, or extreme allergic reaction). In yet another example, the extent is expressed as an expected change in temperature (e.g., the maximum change that is measured at the nasal area) or using a temporal value (e.g., the time it took the increase to occur or the expected time until the temperature at the nasal area will return to normal). In still another example, the extent is determined based on the rate of change in temperature, such that the larger the increase for a given period of time (e.g., five minutes), the more severe the allergic reaction may be considered. And in still another example, the extent of the allergic reaction is a value that is indicative of the area under the curve of the temperature change the ROI over time. Thus, a stronger allergic reaction may, in some cases, correspond to a larger area under the curve. In some embodiments, the circuit provides one or more of the values mentioned above as an output indicative of the extent of the allergic reaction, based on an input that comprises TH_{N}.

In some embodiments, an indication indicative of the extent of the allergic reaction is provided to the user and/or to a third party such as an entity related to the user (e.g., a person or a software agent operating on behalf of the user) or an entity that may provide medical assistance to the user. In one example, the indication may be indicative of the onset of the allergic reaction and/or describe the extent of the allergic reaction (e.g., using one or more of the values described above). Optionally, the indication may be indicative of certain steps that the user should take in order to address the allergic reaction. For example, the indication may suggest the user take a certain dosage of medicine (e.g., an antihistamine), that the user should leave the area (e.g., if outdoors), and/or that the user should seek medical assistance.

There are various ways the indication may be provided to the user. In one example, the frame may be part of a head-mounted system (HMS) that has a display, earphones, and/or other output means (e.g., blinking lights or vibrations), and the indication is provided by the HMS. In another example, the circuit forwards the indication (e.g., via wireless communication) to a device of the user such as a smartphone or a smartwatch and the device provides the indication by alerting the user (e.g., via flashing lights, vibrations, and/or sounds).

The system described above may be utilized, in some embodiments, to identify potential allergens that may be the cause of the rise of the temperature at the ROI. Optionally, the circuit is further configured to identify a potential allergen by estimating the time of exposure to the allergen from a graph exhibiting deviation over time of mean nasal temperature from baseline, and analyzing the items consumed and/or exposed to by the user at that time in order to identify the potential allergen. Optionally, the system is further configured to alert the user about the potential allergen. Optionally, the system is further configured to store in a database plurality of potential allergens identified based on graphs exhibiting deviation over time of mean nasal temperature from baseline. In some embodiments, the system includes a camera mounted to the frame, which is configured to capture the items consumed by the user. Optionally, the system is further configured to show the user an image of the item with the potential allergen.

There are many systems known in the art that may be utilized to monitor what substances a user was exposed to and/or what substances a user consumed. For example, systems that may be utilized to determine what the user ate or drank are described in the patent application US 20110318717 (Personalized Food Identification and Nutrition Guidance System), the U.S. Pat. No. 9,053,483 (Personal audio/visual system providing allergy awareness), the U.S. Pat. No. 9,189,021 (Wearable food nutrition feedback system). Additionally, obtaining indications of possible allergens to which the user was exposed is described in the U.S. Pat. No. 9,000,933 (Automated allergy alerts).

In some embodiments, determination of the extent of the allergic reaction, as described above, may be utilized in the context of allergen challenge tests. For example, the system may be configured to receive an indication of when at least one of a non-invasive intranasal histamine and allergen challenge is performed, and to estimate effects of the histamine or allergen challenge in the tissues, based on increase in nasal temperature. In one example, this involves utilizing the change in TH_{N}, induced by the histamine provocation, as a marker of the intensity of the actions of histamine in the nose. In another example, this may involve utilizing the change in TH_{N}, induced by the allergen challenge, as a marker of the intensity of the actions of the allergen challenge in the nose. Additional examples and discussion regarding allergen challenge tests are provided in the reference Larbig, M., Stamm, H., Hohlfeld, J., & Krug, N. (2003, June), “Levocetirizine but not desloratadine inhibits histamine-induced changes of nasal temperature measured by facial thermography: a pilot study”, In 22nd Congress of the European Academy of Allergy and Clinical Immunology.

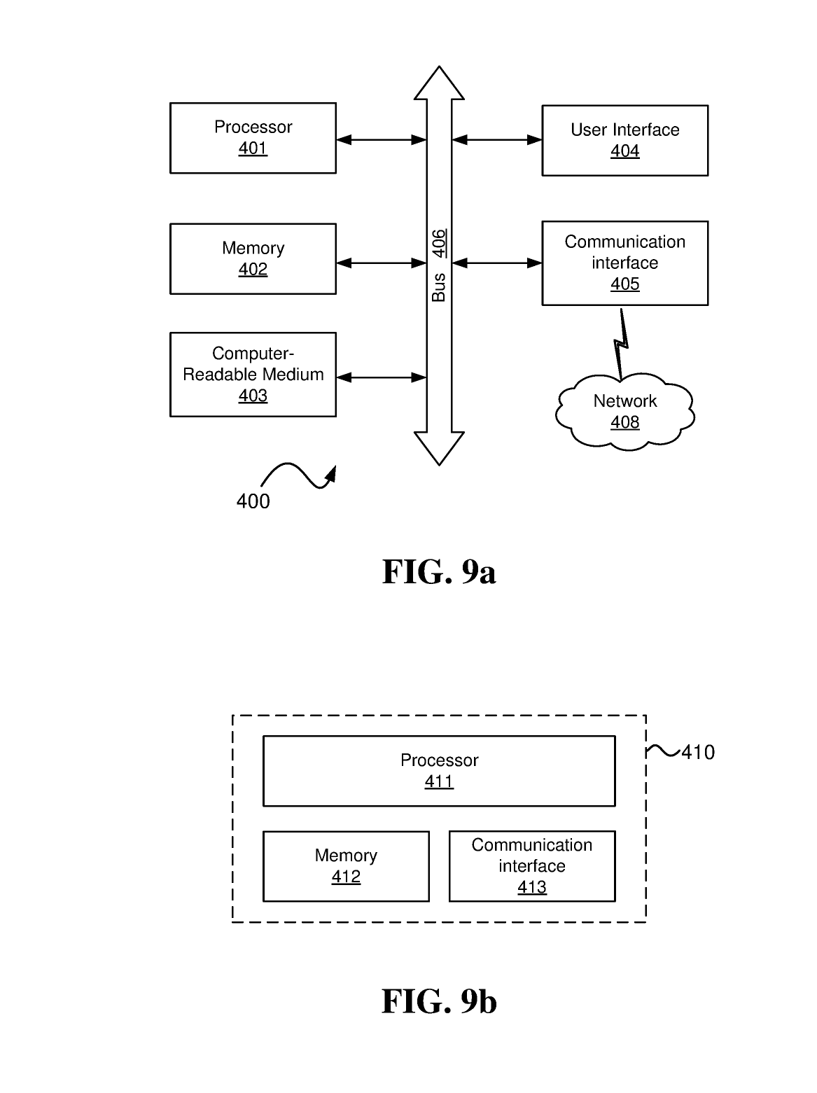

Following is a description of steps that may be performed in various methods involving detecting an allergic reaction. The steps described below may, in some embodiments, be part of the steps performed by an embodiment of a system described above, such as a system modeled according to one of FIG. 1a to FIG. 1b, which includes a frame, a thermal camera that takes thermal measurements of at least part of the nasal area, and a circuit. In some embodiments, instructions for implementing a method described below may be stored on a computer-readable medium, which may optionally be a non-transitory computer-readable medium. In response to execution by a system including a processor and memory, the instructions cause the system to perform operations that are part of the method. Optionally, each of the methods described below may be executed by a computer system comprising a processor and memory, such as the computer illustrated in FIG. 9a or FIG. 9b.

In one embodiment, a method for detecting an allergic reaction of a user includes at least the following steps:

In Step 1, receiving, by a system comprising a circuit, thermal measurements of at least part of the user's nose (TH_{N}). The measurements are taken by a thermal camera weighing less than 5 g, which is physically coupled to the frame worn on the user's head and is located less than 10 cm away from the user's face. Optionally, the thermal camera is based on at least one of the following uncooled sensors: a thermopile, a pyroelectric, and a microbolometer. Optionally, the thermal camera is not in physical contact with the nose, and remains pointed at the nose when the user's head makes angular movements also above 0.1 rad/sec.

In Step 2, determining, based on TH_{N}, whether there is an increase in temperature in the nasal region of the user reaches a threshold. For example, the threshold may be a 0.5° C., 0.8° C., 1.0° C., or some other value greater than 0.5° C. and lower than 2.0° C.

And in Step 3, responsive to a determination that the increase in temperature in the nasal region of the user reaches the threshold, generating an indication indicative of an onset of an allergic reaction of the user. Optionally, indication is generated if the increase in temperature occurs within a certain period of time, such as within 2 minutes, 5 minutes, 10 minutes, 15 minutes, 20 minutes, 25 minutes, 30 minutes, or within some other period of time greater than 2 minutes and lesser than two hours. In one example, the threshold corresponds to an increase of at least 0.5° C., and the indication is generated responsive to determining that the increase occurred during a period of time that is shorter than 10 minutes. In another example, the threshold corresponds to an increase of at least 0.8° C., and the indication is generated responsive to determining that the increase occurred during a period of time that is shorter than 30 minutes.

In one embodiment, the method described above includes a step of determining the extent of the allergic reaction based on at least one of the following values: a magnitude of the increase in the temperature in the nasal region, a rate of the increase to the temperature in the nasal region, a duration within which the threshold was reached. Optionally, the indication is indicative of the extent of the allergic reaction. For example, the indication may be indicative of the maximum expected temperature difference, the duration of the reaction, and/or a value indicative of the severity of the reaction. Additional detail regarding determining the extent of the reaction are given further above.

In one embodiment, the method described above includes a step of identifying a potential allergen by estimating a time of exposure to the allergen from a graph exhibiting deviation over time of mean nasal temperature from baseline, and analyzing items to which the user was exposed at that time in order to identify the potential allergen. Optionally, the method also includes a step of utilizing an image taken by a camera mounted to the frame in order to display the potential allergen to the user.

The following is a discussion regarding properties of the thermal camera, and in particular, the accuracy of its measurements. It is noted that although the following discussion is presented for the sake of brevity in conjunction with the above embodiments involving systems for determining an extent of an allergic reaction, this discussion is relevant to many of the disclosed embodiments herein, which may involve detection of other types of affective responses (e.g., stress).

The prior art perceived a need for expensive thermopiles with an accuracy typically required for medical applications, i.e., having temperature measurement accuracy of ±0.2° C., ±0.1° C., or even better, in order to measure physiological responses with accuracy of ±0.2° C., ±0.1° C. or even better. Whereas the inventors eliminated the need for using such expensive thermopiles to measure physiological responses with accuracy of ±0.2° C., ±0.1° C. or even better.

Systems with plurality of expensive thermopiles with an accuracy typically required for medical applications may be too expensive to be afforded by the average person, and the inventors' insight was contrary to the understandings and expectations of the art that required the use of sensors having temperature measurement accuracy that is equal or better than the expected temperature changes associated with the physiological response to be measured.

It is noted that sentences such as “temperature change accuracy better than ±0.1° C.” mean that the difference between the temperature change of the ROI and the temperature change measured by a sensor pointed at the ROI is less than ±0.1° C. For example, when the temperature of the ROI changes from 37.56 to 37.82° C. and a thermopile pointed at the ROI measures a change from 38.66 to 37.93° C., then the thermopile's temperature measurement accuracy is 1.1° C. while the thermopile's temperature change accuracy is 0.01° C.

It is specifically noted that although many of the disclosed embodiments work well with inexpensive thermopiles that provide temperature measurement accuracy above ±0.50° C., these embodiments can also utilize the expensive thermopiles, which have an accuracy that is typically required in medical applications, to achieve even better results.

In some embodiments, the thermal camera is based on at least one of the following uncooled sensors: a thermopile, a pyroelectric, and a microbolometer. Optionally, the thermal camera comprises a single sensing element. Alternatively, the thermal camera may comprise multiple sensing elements.

In different embodiments, the thermal camera may take measurements with different accuracies for measurements of temperature (T) vs. measurements of the temperature change (ΔT). Optionally, in these embodiments, the circuit utilizes ΔT to determine the physiological response (e.g., determine the extent of an allergic reaction or determine a stress level). For example, in one embodiment, the thermal camera provides temperature measurement accuracy better than ±1.0° C. and provides temperature change (ΔT) accuracy better than ±0.10° C. In another embodiment, the thermal camera provides temperature measurement accuracy better than ±0.50° C. and provides temperature change (ΔT) accuracy better than ±0.080° C. And in yet another embodiment, the thermal camera provides temperature measurement accuracy better ±0.20° C. and provides temperature change (ΔT) accuracy better than ±0.040° C.

In different embodiments, the system's nominal measurement error of the temperature at the ROI (ERR_{TROI}) may be greater than the system's nominal measurement error of the temperature changes at the ROI (ERR_{ΔTROI}). For example, the measurement error of the temperature at the nasal region might be greater than the measurement error of the change to the temperature in the nasal region. In one embodiment, ERR_{TROI} is at least twice ERR_{ΔTROI} when the user's head makes angular movements at a rate above 0.1 rad/sec. In this embodiment, the circuit is able to identify affective response that causes a temperature change at the ROI, which is between ERR_{ΔTROI} and ERR_{TROI}. In another embodiment, ERR_{TROI} is at least five times ERR_{ΔTROI} when the user's head makes angular movements at a rate above 0.5 rad/sec. In this embodiment, the circuit is able to identify affective response that causes a temperature change at the ROI, which is between ERR_{ΔTROI} and ERR_{TROI}.

Another application that involves thermal measurements of the face (and more specifically the nasal region) is the estimation of the level of stress a user is under, as described in the following embodiment. In one embodiment, a system configured to estimate stress level of a user wearing a head-mounted system (HMS) includes at least a frame, a thermal camera, and a circuit.

The frame is configured to be worn on the user's head and the thermal camera, which weighs below 5 g, is physically coupled to the frame and located less than 10 cm away from the user's face. The thermal camera is configured to take thermal measurements of a region of interest (TH_{ROI}), where the ROI covers at least part of the area around the user's nose. Optionally, the thermal camera is located less than 3 cm away from the user's face and weighs below 1 g. Optionally, the system does not occlude the ROI.

The measurements TH_{ROI} may be represented as time series data, which includes values indicative of the temperature (or change to temperature) at an ROI that includes part of the user's nose at different times. In different embodiments, these measurements may be taken at different intervals, such as a few times a second, once a second, every few seconds, once a minute, and in some cases, every few minutes.

One example of the ROI around the nostrils is described in the reference Shastri, D., Papadakis, M., Tsiamyrtzis, P., Bass, B., & Pavlidis, I. (2012), “Perinasal imaging of physiological stress and its affective potential”, Affective Computing, IEEE Transactions on, 3(3), 366-378.

It is to be noted that sentences such as “the area around the user's nose” refer to the area of the nose/nasal and up to 3 cm from the nose, where the exact area depends on the application and the physiological response to be measured. Thus, while in some embodiments, a system that is used to estimate stress may take measurements from the same ROI described above for the system that detects an allergic reaction, in other embodiments, these ROIs may be slightly difference. Guidance towards determining the locations of the ROIs for the various applications is provided in the references cited for each application and/or the description of the embodiments given herein.

The circuit is configured to estimate the stress level based on TH_{ROI}. The circuit may be any of the various types of circuits mentioned in this disclosure, e.g., it may be a processor, an ASIC, or an FPGA. In one example, the circuit is the circuit 16 described in FIG. 1a. In some embodiments, the circuit may be coupled to the frame and/or to an HMS of which the frame is a part. In other embodiments, the circuit may belong to a device carried by the user (e.g., a processor of a smartwatch or a smartphone).

In some embodiments, estimating the stress level is done by a circuit that is remote from the user. For example, the circuit may belong to cloud-based server, which receives TH_{ROI}, processes those values, and returns a result to the user (e.g., a value indicative of the stress level).

Determining the stress level may be done by examining various properties of TH_{ROI}, which may involve For example, an onset may be detected if the rise in the temperature of an ROI in the nasal area and/or the mouth exceeds a certain threshold value such as, 0.4° C., 0.8° C., 1.0° C., or some other value greater than 0.4° C. and lower than 2.0° C. Optionally, the onset is detected if the rise exceeding the certain value occurs within a short period of time, such as 2 minutes, 5 minutes, 10 minutes, 15 minutes, 20 minutes, 25 minutes, 30 minutes, or some other period of time greater than 2 minutes and lesser than two hours.

When determining the stress level, in some embodiments, additional inputs other than TH_{ROI} may be utilized. In one example, measurements of the environment taken with sensors may be utilized for this purpose. For example, the measurements may correspond to environmental parameters such as temperature, humidity, UV radiation levels, etc. In another example, the additional inputs may comprise values indicative of activity of the user, such as inputs from movement sensors and/or accelerometers. In still another example, the additional inputs may comprise temperature values of the user's body and/or cutaneous temperatures of other regions of the user's face and/or body (e.g., regions other than the nasal and/or mouth areas).

The various inputs described above may be utilized, in some embodiments, by the circuit to make more accurate estimations of the stress level. For example, these inputs may be utilized in order to rule out false positives in which the ROIs may display an increase in temperature than is not due to an allergic reaction, such as temperature increases due to the environment (e.g., when exposed to the sun) and/or temperature increases due to the user's activity (e.g., while running or exercising).

In some embodiments, estimating, based on TH_{ROI}, the stress level, may be done utilizing a machine learning-based model. In such embodiments, the circuit may compute various features derived from TH_{ROI} (e.g., values of the temperature or change in temperature at different preceding times, and/or the change in temperature relative to various preceding points in time), and utilize the model to generate an output indicative of the stress level. Optionally, features may include values derived from one or more of the additional input sources described above (e.g., environmental measurements, user activity signals, and/or temperature measured at other reasons).

In some embodiments, the model is generating based on labeled training data that includes samples including samples that each include feature values derived from values of TH_{ROI} and labels indicative of the stress. Optionally, some labels may be provided by the user to samples generated from measurements of the user (thus, the model may be considered a personalized model of the user).

In one embodiment, the system optionally includes a user interface configured to alert the user when the stress level reaches a predetermined threshold.

In some embodiments, one or more additional thermal cameras may be utilized for the detection of stress. For example, in one embodiment, more than half of the ROI covers the right side of the area around the user's nose. In this embodiment, the system may further include a second thermal camera, physically coupled to the frame, configured to take thermal measurements of a second ROI (TH_{ROI2}), where more than half of the ROI₂ covers the left side of the area around the user's nose. Optionally, the first and second thermal cameras are configured to provide, to a circuit, measurements of temperatures at ROI and ROI₂, denoted T_{ROI} and T_{ROI2}, respectively. In this case, the circuit may be configured to: calculate a change-to-temperature-at-ROI (ΔTROI) based on TROT, calculate a change-to-temperature-at-ROI₂ (ΔT_{ROI2}) based on T_{ROI2}, and to utilize ΔT_{ROI1} and ΔT_{ROI2} to identify the stress level. Additionally or alternatively, the first and second thermal cameras are configured to provide, to a circuit, measurements of temperatures at ROI and ROI₂, denoted T_{ROI} and T_{ROI2}, respectively. And in this case, the circuit may be configured to: calculate a difference between T_{ROI} and T_{ROI2} at time m (denoted ΔTₘ), calculate a difference between T_{ROI} and T_{ROI2} at time n (denoted ΔTₙ), and identify the stress level based on a difference between ΔTₘ and ΔTₙ.

The difference between the right and left sides around the user's nose may be used to detect asymmetric patters that characterize the user (such as right side being a bit hotter when the user reaches a certain stress level), and/or detect interference from the environment (such as direct sunlight on the right side, which makes it a bit hotter).

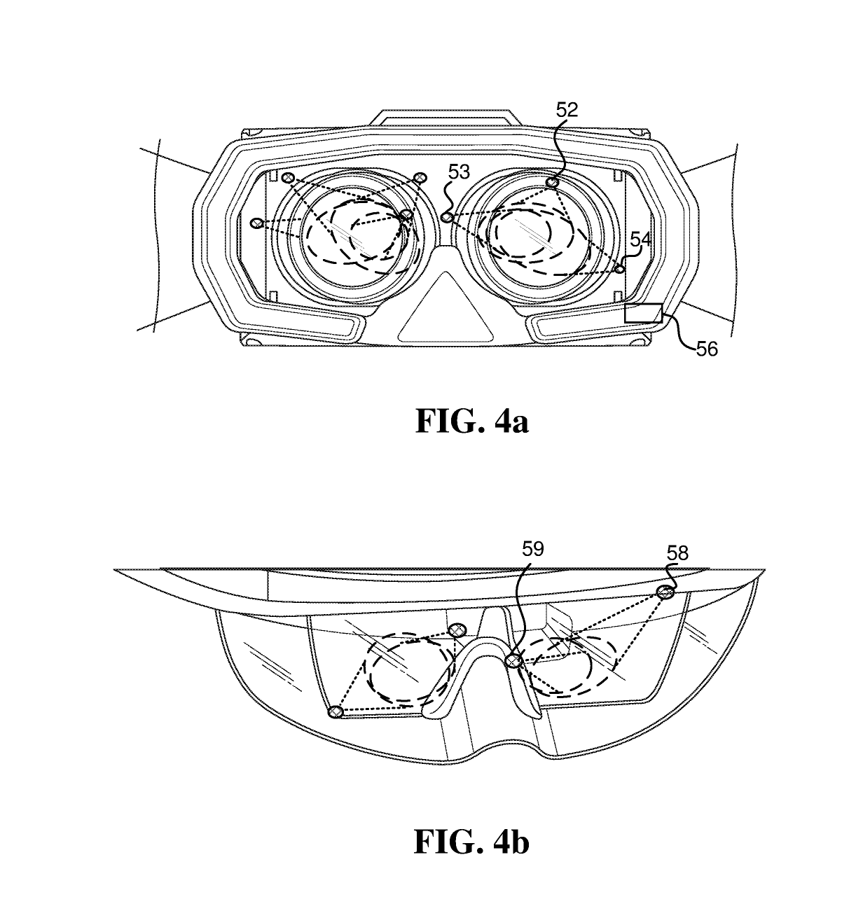

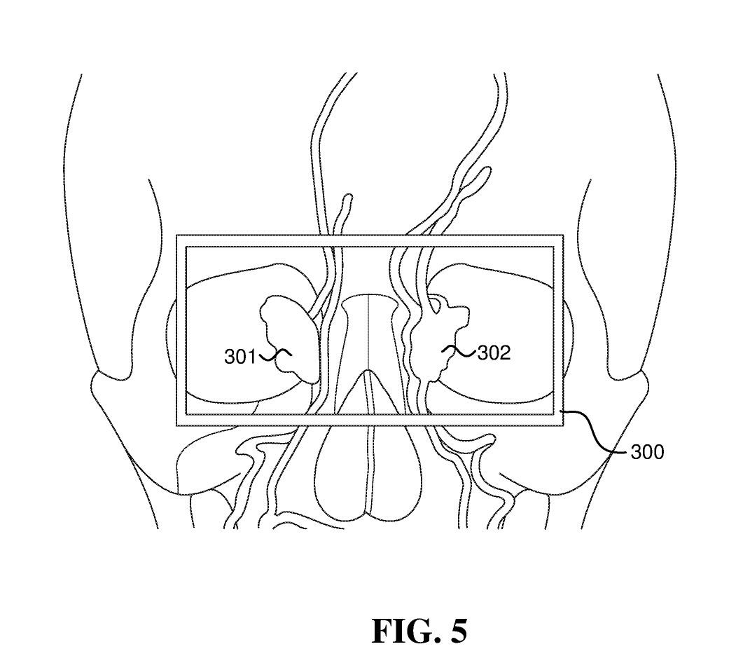

In addition to the nasal and mouth regions, another region on the face in which thermal measurements can be indicative of stress in the periorbital region. FIG. 4a and FIG. 4b illustrate various potential locations to connect thermal cameras to various head mounted display frames in order to have at least some of the periorbital ROI within the field of view of one or more of the thermal cameras. Because the thermal cameras are located close to the ROI, they can be small, lightweight, and may be placed in many potential locations having line of sight to the respective ROIs.

The periorbital region of the user's face is discussed, for example, in the reference Tsiamyrtzis, P., Dowdall, J., Shastri, D., Pavlidis, I. T., Frank, M. G., & Ekman, P. (2007), “Imaging facial physiology for the detection of deceit”, International Journal of Computer Vision, 71(2), 197-214. FIG. 5 illustrates the periorbital ROI, schematically represented by rectangle 300. Regions 301 and 302, referred to as the conduits in the eye corners, schematically represent about 10% of the warmest area within the periorbital ROI, which may be sufficient to detect the “fight or flight” syndrome/response during stress (also known as fight or flight syndrome). The reference Pavlidis, I., Levine, J., & Baukol, P. (2000), “Thermal imaging for anxiety detection”, In Computer Vision Beyond the Visible Spectrum: Methods and Applications, 2000. Proceedings. IEEE Workshop on (pp. 104-109), also shows the periorbital region, together with the nasal area, right and left cheeks, chin area, and the neck area.

Referring back to FIG. 4a, the figure illustrates one embodiment of a wearable system, such as a head mounted system (HMS), configured to estimate a stress level. The system includes a frame, a thermal camera and circuit. The frame is configured to be worn on a user's head. The thermal camera is physically coupled to the frame, located less than 10 cm away from an eye of the user, and takes thermal measurements of a region of interest (TH_{ROI}) that covers at least part of a periorbital region of the eye. Locations 52, 53, and 54 in FIG. 4a illustrate possible positions for locating tiny thermal cameras for measuring the periorbital region around the right eye. The circuit 56, which may by wearable by the user or non-wearable, is configured to estimate the stress level of the user based on changes to temperature of the periorbital region received from the thermal camera. Optionally, the circuit comprises at least one of the following: a differential amplifier coupled to the frame, an analog circuit coupled to the frame, a processor physically coupled to the frame, a processor worn by the user, a processor of a smartphone belonging to the user, a processor in a server accessed via a communication network, and a processor in a cloud computer accessed via the Internet.

FIG. 4b illustrates additional locations (Locations 58 and 59) for tiny thermal cameras for measuring the periorbital region around the left eye, when the HMS is viewed from the outer direction in.

In one embodiment, the delay between a stressful event and its manifestation on the at least part of the periorbital region is less than one minute, and most of the manifestation diminishes within less than five minutes after the stressful event is over.

In one embodiment, the system described above optionally includes a display, physically coupled to the frame, which is configured to present digital content to a user who wears the display. The display does not occlude the thermal camera from measuring the at least part of the periorbital region of the user's eye. Optionally, the system includes a computer configured to change the digital content presented to the user based on the estimated stress level.

In another embodiment, the system optionally includes an eye tracking module coupled to the frame and configured to track gaze of the user. In this embodiment, the HMS is an optical see through head mounted display configured to operate in cooperation with: a second camera configured to capture images of objects the user is looking at, and a processor configured to match the objects the user is looking at with the estimated stress levels.

In yet another embodiment, the system optionally includes a display coupled to the frame and configured to present video comprising objects, and an eye tracking module coupled to the frame and configured to track gaze of the user. In this embodiment, the HMS is configured to operate in cooperation with a processor configured to match the objects the user is looking at with the estimated stress levels.

In still another embodiment, the system may optionally include a user interface configured to notify the user when the stress level reaches a predetermined threshold. Optionally, the user interface utilizes at least one of an audio indication and visual indication to notify the user. Additionally or alternatively, the greater the change to the temperature of the periorbital region, the higher the stress level, and the indication is proportional to the stress level. Optionally, the notification may encourage the user not to engage in negative behavior (e.g., lying or cheating) by exposing the user to evidence that engaging the negative behavior increases stress (e.g., evidence based on measurements of the user) and/or reminding the user of the negative outcomes that may be caused by the negative behavior.

In order to assist the user in relieving stress, in some embodiments, the system described above includes a computer and a user interface configured to suggest the user to participate in stress relieving activities when the stress level reaches a first predetermined threshold, such as practice yoga (e.g., pranayama), engage in brainwave stimulation-based entrainment, physical exercise, and/or hear positive encouraging statements. Optionally, the computer is further configured to suggest the user to stop the activity when the stress level gets below a second predetermined threshold.

In one embodiment, the system may optionally include: a display configured to show the user a video comprising objects, and a documenting module configured to store the estimated stress level associated with the viewed objects.

Alertness, anxiety, and even fear appear to accompany people that are involved in illegal activities at the time of their action. Since those symptoms are produced by the sympathetic system, they cannot be totally controlled, and thus constitute a powerful biometric that is difficult to conceal. This biometric can provide valuable clues to security systems of critical/sensitive facilities/data about potential suspects immune to identification biometrics, such as first time offenders.

When a user experiences elevated feelings of alertness, anxiety, or fear, increased levels of adrenaline regulate blood flow. Redistribution of blood flow in superficial blood vessels causes abrupt changes in local skin temperature that is readily apparent in the user's face where the layer of flesh is very thin. The human face and body emit both in the mid-infrared (3-5 μm) and far-infrared (8-12 μm) bands, thus mid-infrared and far-infrared thermal sensors can sense this temperature variations in the face and trigger a process for detecting the illegal activity.

In one embodiment, a user is permitted to access sensitive data only through an HMD equipped with a thermal camera that measures temperature variations on the user's face while he/she is accessing the sensitive data. This way the user is under surveillance each time he/she accesses the sensitive data, and optionally there is no way for the user to access the sensitive data without being monitored by the system.

The following is a description of one embodiment of such a system, which is configured to detect an irregular activity. The system includes at least a head mounted display (HMD) and a processor. The HMD includes a frame, a display module, and a thermal camera. The thermal camera weighs less than 5 g, is physically coupled to the frame, and located less than 10 cm away from the user's face. The thermal camera is configured to take thermal measurements of a region of interest (TH_{ROI}) on the user's face. Optionally, the thermal camera comprises an uncooled thermal sensor.

The processor is configured to: calculate a baseline thermal profile for the user based on values of TH_{ROI} taken while the user watches baseline sensitive data presented by the HMD, calculate a certain thermal profile for the user based on values of TH_{ROI} taken while the user watches certain sensitive data presented by the HMD, and issue an alert when the difference between the certain thermal profile and the baseline thermal profile reaches a predetermined threshold.

In one embodiment, the alert relates to a process for detecting an illegal activity. Optionally, the delay between the time of performing the illegal activity and the time of reaching the predetermined threshold is less than two minutes. Optionally, the material accessed the belongs to an organization, the user is an employee of the organization, and the system helps in preventing illegal activities of employees related to sensitive data. In another embodiment, the alert relates to fatigue or job burnout of the user. In this embodiment, the processor is further configured to utilize the alert to estimate job burnout, such that the greater the difference between the certain thermal profile and the baseline thermal profile, the worse is the job burnout.

In addition to issuing the alert described above, in one embodiment, the processor is further configured to detect that the user moved the HMD while being exposed to the certain sensitive data, and not allow the user to perform a certain transaction related to the certain sensitive data. In one example, the certain transaction comprises at least one of the following transactions: copying, reading, and modifying the certain sensitive data. In another example, the certain sensitive data relates to money, and the certain transaction comprises electronic funds transfer from one person or entity to another person or entity. In another embodiment, the processor is further configured to: detect that the user moved the HMD while being exposed to the certain sensitive data, mark as suspicious the relationship between the user and the certain sensitive data, and issue a security alert after detecting that the user moved again the HMD while being exposed to another sensitive data that is of the same type as the certain sensitive data.

There may be various possible ROIs that may be utilized in different embodiments. In one embodiment, the ROI covers at least part of periorbital region of the user's face. In another embodiment, the ROI covers at least part of the user's nose. And in still another embodiment, the ROI covers at least part of the user's forehead.

In different embodiments, TH_{ROI} may include different types of values. In one example, TH_{ROI} expresses temperature at the ROI, and the baseline thermal profile expresses ordinary temperature at the ROI while the user is exposed to sensitive data. In another example, TH_{ROI} expresses temperature change at the ROI, and the baseline thermal profile expresses ordinary temperature changes at the ROI around the time of switching from being exposed to non-sensitive data to being exposed to sensitive data. And in yet another example, TH_{ROI} expresses temperature change at the ROI, and the baseline thermal profile expresses ordinary temperature changes at the ROI around the time of switching from being exposed to sensitive data to being exposed to non-sensitive data.

In one embodiment, the processor is further configured to issue a second alert when the difference between the certain thermal profile and the baseline thermal profile reaches a second predetermined threshold that is greater than the predetermined threshold. Optionally, the irregular activity is illegal activity, and the probability to detect occurrence of the illegal activity is at least twice higher when reaching the second predetermined threshold than reaching the predetermined threshold.

In some cases, it may be useful to compare close events because the shorter the time between watching the baseline sensitive data and watching the certain sensitive data, the smaller the negative effect of environmental changes and normal physiological changes may be. In one example, the user watches the certain sensitive data immediately before and/or after watching the baseline sensitive data. In another example, the user watches the certain sensitive data within less than 5 minutes before and/or after watching the baseline sensitive data. In still another example, the user watches the certain sensitive data within less than 15 minutes before or after watching the baseline sensitive data.

It is to be noted that when the user observes data over period of time, in some embodiments, each segment of data (e.g., data observed during a certain span of a few minutes) may serve both as baseline sensitive data (for a certain evaluation) and as the certain sensitive data (for another evaluation).

The environment in which the user views data may influence the user's thermal profile. Therefore, in some embodiments, the processor may be further configured to receive characteristics of the environment the user is in while watching the certain sensitive data, and further configured to select as the baseline an event in which the user watched the baseline sensitive data while being in a similar environment. In one example, the difference in ambient temperatures of similar environments is less than 2° C. In another example, the difference in humidity of similar environments is less than 5%. In still another example, the difference in oxygen percentage in the air of similar environments is less than 2%.

Thermal measurements can be utilized to identify an object that agitates a user. Following is an example embodiment of such a system. In one embodiment, the system includes at least a frame, an eye tracking module, a thermal camera, and a processor.

The frame is configured to be worn on a user's head, and the eye tracking module coupled to the frame and configured to track the gaze of the user while watching a video comprising objects. At least some of the objects associated with expected attention levels obtained from saliency mapping.

The thermal camera, which weighs less than 5 g, is physically coupled to the frame and pointed at a region of interest (ROI) on the user's face. The thermal camera is configured to take thermal measurements of the ROI (TH_{ROI}). Optionally, thermal camera is not in physical contact with the ROI, is located outside the exhale streams of the mouth and nostrils, and remains pointed at the ROI when the user's head makes angular movements also above 0.1 rad/sec. There may be various possible ROIs that may be utilized in different embodiments. In one example, the ROI covers at least part of periorbital region of the user's face. In another example, the ROI covers at least part of the user's nose. And in still another example, the ROI covers at least part of the user's forehead.

The processor is configured to: estimate stress level of the user based on TH_{ROI}, calculate level of mismatches between attention levels in the at least some of the objects that are based on the user's gaze and the corresponding values based on the saliency mapping, and to generate, based on the stress level and the level of mismatches, an output indicative of the probability that the video includes an object that agitates the user. Optionally, the output is indicative of negative feelings related to at least one of an object and a situation presented in the video. Additionally or alternatively, the output may be indicative of an extent to which the user has something to hide.

In one embodiment, the probability is proportional to the product of the stress level and the level of mismatch. Thus, the higher the mismatch and/or the higher the stress level, the higher the probability that the video contained an object that agitates the user. In one example, the agitating object causes the user to stare at it longer than expected according to the saliency mapping. In another example, the agitating object causes the user to stare at it for a shorter period than expected according to the saliency mapping (e.g., in a case where the user is freighted and/or disgusted by the object).

Herein, “saliency mapping” may refer to one or more of various techniques that may be used to assign to visual objects, in images and/or video, values that represent an expected attention level in the objects. For example, an object that stands out more, e.g., due to a color difference with respect to the background and/or movement compared to a relatively stationary background, is expected to correspond to a higher attention level than an object that does not stand out.

There are various ways in which saliency mapping may be performed. In some embodiments, an algorithmic approach is utilized to calculate saliency values for objects. Some examples of various approaches known in the literature include approaches described in Spain, M. & Perona, P. (2011), Measuring and Predicting Object Importance, International Journal of Computer Vision, 91 (1). pp. 59-76. In another example, user interest in objects may be estimated using various video-based attention prediction algorithms such as the one described in Zhai, Y. and Shah, M. (2006), Visual Attention Detection in Video Sequences Using Spatiotemporal Cues, In the Proceedings of the 14th annual ACM international conference on Multimedia, pages 815-824, or Lee, W. F. et al. (2011), Learning-Based Prediction of Visual Attention for Video Signals, IEEE Transactions on Image Processing, 99, 1-1.

In other embodiments, the saliency mapping involves utilizing measurements (e.g., from eye tracking) in order to determine what interests users and/or how long the gaze at various objects. Optionally, the measurements may include previous measurements of the user related to the objects. Additionally or alternatively, the measurements may include measurements of other users.

A system, such as the one described above, may be utilized for various security-related applications. In one embodiment, the processor is further configured to identity an object whose assigned stress level is above a predetermined threshold as a suspicious object. Optionally, the processor is further configured to indicate to an interrogator to focus an interrogation on the suspicious object.

The following is a description of an embodiment of method for a security-related application for identifying a suspicious object viewed by an interrogee. In one embodiment, the method includes at least the following steps:

In Step 1, capturing images of an interrogee when standing or walking, with or without his/her belongings.

In Step 2, generating a first video of the interrogee and his/her belongings;

In Step 3, taking, while the interrogee watches the first video, thermal measurements of a region of interest (ROI) and obtaining eye tracking data indicative of where the interrogee is looking. The ROI comprises of at least a portion of at least one of the following regions on the face of the interrogee: the periorbital region, the nose, and the forehead.

In Step 4, identifying a suspicious object in the first video. Optionally, the suspicious object relates to at least one of the interrogee's body, closes, and belongings.

In Step 5, generating a second video that emphasizes the suspicious object more than the first video. Optionally, the second video emphasizes the suspicious object more than the first video by focusing the scene of the second video on the suspicious object.

In Step 6, taking, while the interrogee watches the second video, thermal measurements of the region of interest and eye tracking data indicative of where the interrogee is looking.

And in Step 7, issuing an alert when the absolute value of the change in the thermal measurements, while looking at the suspicious object, is more than a predetermined threshold above the absolute value of the change in the thermal measurements while not looking at the suspicious object. Optionally, the predetermined threshold is above at least one of the following temperature changes: 0.05° C., 0.1° C., 0.2° C., and 0.4° C.

In one embodiment, the second video switches at least 3 times between the suspicious object and a non-suspicious object, and the method further comprises as step of comparing the thermal measurements of at least one of the ROIs at a time corresponding to viewing the suspicious object with the thermal measurements of the same ROI corresponding to viewing of the non-suspicious object, and calculating a probability that the interrogee has something to hide based on the comparison.

In one embodiment, the first and second videos are presented by a head mounted display, and the thermal camera is coupled to the head mounted display. Optionally, the thermal camera is coupled to the head mounted display at a position that is less than 15 cm away from the interrogee's head. Optionally, the interrogee's ear is not in the field of view of the thermal camera.

The Face, Head-Mounted Systems, and Thermal Cameras

The following is a discussion describing aspects that may be relevant to the various embodiments described in this disclosure. Some aspects described below involve descriptions of the human face and face-related nomenclature used herein. Additional aspects described below involve various properties and configurations of head-mounted systems (HMSs) that may be utilized in some of the embodiments in this disclosure. And additionally, some aspects described in detail below involve various properties and configurations of thermal cameras that may be used in different embodiments.

Various embodiments described herein involved taking thermal measurements of a Regions Of Interest (ROIs) on a user's face. The following is a discussion regarding facial anatomy and nomenclature that may be used to define the various facial regions covered by ROIs and/or locations of thermal cameras, in embodiments described herein.

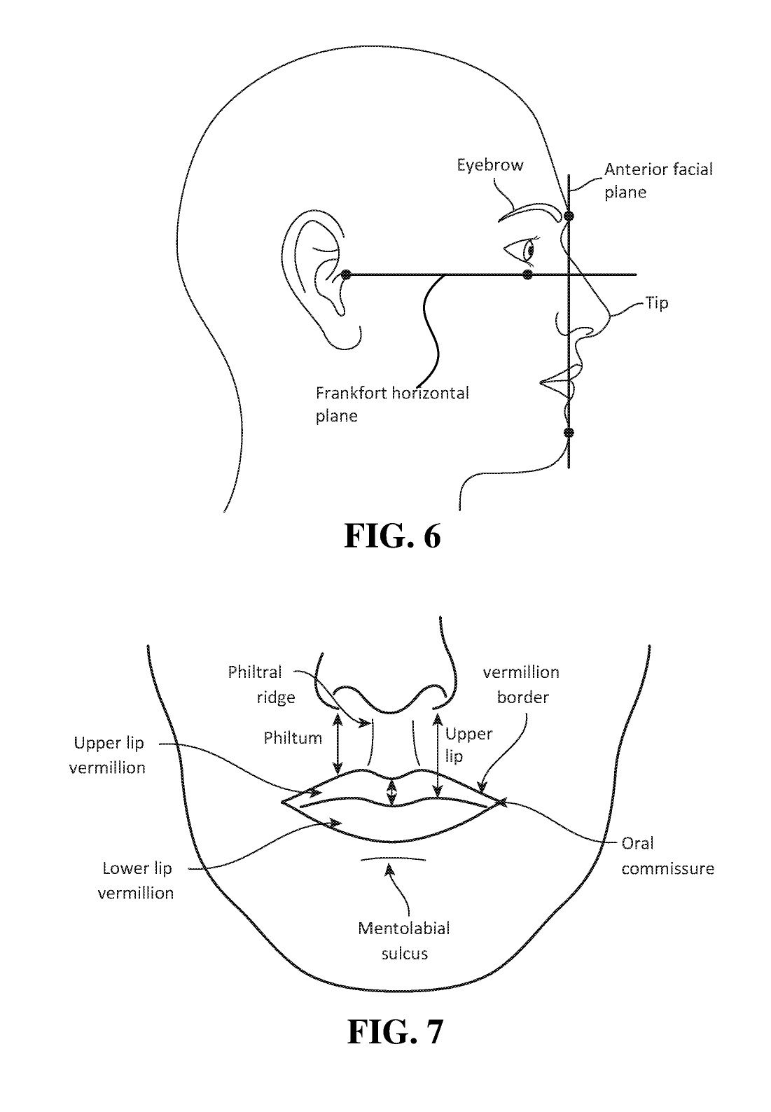

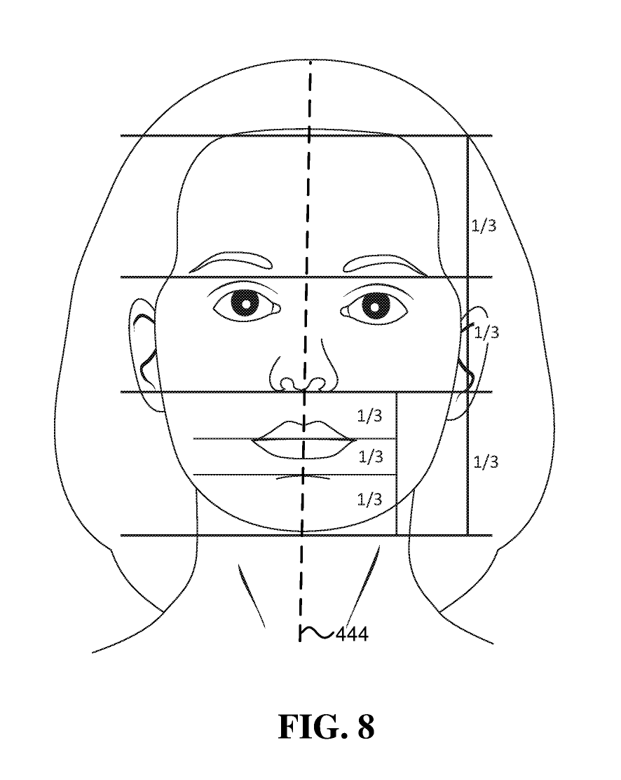

FIG. 6 illustrates the Frankfort horizontal plane and anterior facial plane as these terms are used herein. A line from the superior aspect of the external auditory canal to the most inferior point of the orbital rim creates the Frankfort horizontal plane (known also as the Frankfurt horizontal plane or Frankfort plane). A line from the glabella to pogonion creates the anterior facial plane. FIG. 7 illustrates the upper lip, upper lip vermillion, lower lip vermillion, and the oral commissure, which is the place where the lateral aspects of the vermilion of the upper and lower lips join. FIG. 8 illustrates the horizontal facial thirds. The upper horizontal facial third extends from the hairline to glabella, the middle horizontal facial third extends from glabella to subnasale, and lower horizontal facial third extends from subnasale to menton. The lower horizontal facial third is further divided into thirds: the lower-upper horizontal facial third extends from subnasale to stomion (defines the upper lip), the lower-middle horizontal facial third extends from stomion to the labiomental crease (defines the lower lip), and the lower-lower horizontal facial third extends from the labiomental crease to menton (defines the chin). It is noted that the thirds are usually not equal. Symmetry axis 444 divides the face to the right and left sides.

It is noted that all measurements, notations, planes, angles, distances, horizontal facial thirds, and/or elements of the user's face (such as eyes, nose, lips, eyebrows, hairline) herein refer to a normal, 20 year old, aesthetic human, such as described in Chapter 2, Facial Proportions, by Peter M. Prendergast, in the book “Advanced Surgical Facial Rejuvenation, Art and Clinical Practice”, Editors: Erian, Anthony, Shiffman, Melvin A., Publisher: Springer-Verlag Berlin Heidelberg, 2012. It is further noted that the appearance of the face varies with facial movement, thus, when appropriate according to the context, the positions of the elements of the user's face (such as eyes, nose, lips, eyebrows, hairline), and the distances between various cameras/sensors and the user's face, are usually assessed herein when the user has a relaxed (neutral) face: the eyes are open, the lips make gentle contact, and the teeth are slightly separated. The neck, jaw, and facial muscles are not stretched nor contracted, and the face is positioned using the Frankfort horizontal plane.

The reference Ioannou, S., Gallese, V., & Merla, A. (2014), “Thermal infrared imaging in psychophysiology: potentialities and limits”, Psychophysiology, 51(10), 951-963, provides in Table 1 a useful overview of the direction of temperature variation in various ROIs across emotions, and a useful summary regarding temporal latency of cutaneous temperature change.

Various types of systems and/or hardware configurations may be utilized in embodiments described in this disclosure. Some embodiments involve a Head-Mounted System (HMS) that includes a frame. Optionally, the frame may be similar to a frame of eyeglasses, having extending side arms (i.e., similar to eyeglasses temples). The frame may extend behind a user's ears to secure the HMS to the user. The frame may further secure the HMS to the user by extending around a rear portion of the user's head. Additionally or alternatively, the frame may connect to or be affixed within a head-mountable helmet structure.

Various systems described in this disclosure may include a display that is coupled to a frame worn on a user's head, e.g., a frame of a HMS. In some embodiments, the display coupled to the frame is configured to present digital content, which may include any type of content that can be stored in a computer and presented by the computer to a user. Phrases in the form of “a display coupled to the frame” are to be interpreted in the context of one or more of the following configurations: (i) a frame that is worn and/or taken off together with the display such that when the user wears/takes off the HMS he/she also wears/takes off the display, (ii) a display integrated with the frame; optionally the display is sold together with the HMS, and/or (iii) the HMS and the display share at least one electronic element, such as a circuit, a processor, a memory, a battery, an optical element, and/or a communication unit for communicating with a non-head mounted computer.

Herein a display may be any device that provides a user with visual images (e.g., text, pictures, and/or video). The images provided by the display may be two-dimensional or three-dimensional images. Some non-limiting examples of displays that may be used in embodiments described in this disclosure include: (i) screens and/or video displays of various devices (e.g., televisions, computer monitors, tablets, smartphones, or smartwatches), (ii) headset- or helmet-mounted displays such as augmented reality systems (e.g., HoloLens), virtual reality systems (e.g., Oculus rift, Vive, or Samsung GearVR), and mixed reality systems (e.g., Magic Leap), and (iii) image projection systems that project images on a user's retina, such as: Virtual Retinal Displays (VRD) that create images by scanning low power laser light directly onto the retina, or light-field technologies that transmit light rays directly into the eye.

In one embodiment, a helmet is coupled to the frame and configured to protect the user's scalp. Optionally, the helmet may be is at least one of the following: a sports helmet, a motorcycle helmet, a bicycle helmet, and a combat helmet. Phrases of the form of “a helmet coupled to the frame” are to be interpreted in the context of one or more of the following configurations: (i) a frame that is worn and/or taken off together with the helmet such that when the user wears/takes off the helmet he/she also wears/takes off the HMS, (ii) a frame integrated with the helmet and/or the helmet itself forms the frame; optionally the HMS is sold together with the helmet, and/or (iii) the HMS and the helmet share at least one electronic element, such as an inertial measurement sensor, a circuit, a processor, a memory, a battery, an image sensor, and/or a communication unit for communicating with a non-head mounted computer.

In one embodiment, a brainwave-measuring headset is coupled to the frame and configured to collect brainwave signals of the user. Phrases in the form of “a brainwave-measuring headset coupled to the frame” are to be interpreted in the context of one or more of the following configurations: (i) a frame that is worn and/or taken off together with the brainwave-measuring headset such that when the user wears/takes off the brainwave-measuring headset he/she also wears/takes off the HMS, (ii) a frame integrated with the brainwave-measuring headset and/or the brainwave-measuring headset itself forms the frame; optionally the HMS is sold together with the brainwave-measuring headset, and/or (iii) the HMS and the brainwave-measuring headset share at least one electronic element, such as an inertial measurement sensor, a circuit, a processor, a memory, a battery, and/or a communication unit.

Known systems for analyzing physiological responses based on temperature measurements receive series of thermal images composed of pixels that represent temperature (T) measurements. Measuring the temperature (as opposed to temperature change) is required in order to run a tracker and perform image registration, which compensate for the movements of the user in relation to the thermal camera and brings the images into precise alignment for analysis and comparison.

In one embodiment, a thermal camera (also referred to as a thermal sensor) is coupled to a frame worn on a user's head. In this configuration, the thermal camera moves with the user's head when the head changes its location and orientation in space, and thus there may be no need for a tracker and/or there may be no need for image registration. As a result, it is possible to run the image processing and/or signal processing algorithms on the series of thermal differences (ΔT) measured by each thermal sensing element. Running the image/signal processing algorithms on the measured ΔT increases the accuracy of the system significantly compared to the case where ΔT is derived from images/signals representing temperature measurements (T). Optionally, the temperature change at the ROI over time (ΔTROI) is analyzed in relation to another parameter, such as the stimulus the user is exposed to, and/or other physiological measurements (such as EEG, skin conductance, pulse, breathing rate, and/or blood pressure).

Examples of thermopile sensors that may be useful for at least some of the embodiments herein, optionally with some adaptations, include Texas Instruments “TMP006B Infrared Thermopile Sensor in Chip-Scale Package”, Melexis “MLX90614 family Single and Dual Zone Infra-Red Thermometer in TO-39”, Melexis MLX90614 in TO-46, HL-Planartechnik GmbH “TS118-3 thermopile sensor”, Dexter Research Center, Inc. “DX-0875 detector”, Dexter Research Center, Inc. “Temperature Sensor Module (TSM) with ST60 thermopile and onboard ASIC for amplification, digitizing, temperature compensation and calibration”. When it is assumed that the sensor keeps measuring the same area on the object, these examples of thermopile sensors can provide readings of ΔT, where often the measurement error of ΔT is much smaller than the measurement error of T. Therefore, maintaining the thermal camera pointed at the ROI, also when the user's head makes angular movements, enables at least some of the embodiments to utilize the more accurate ΔT measurement to identify fine physiological responses that may not be identified based on image processing of temperature measurements (T) received from a camera that is not continuously pointed at the ROI (assuming sensors with same characteristics are used in both scenarios). It is noted that each of the above-mentioned thermal sensors weighs below 1 g.

In some embodiments, a thermal camera may operate at a frequency that may be considered relatively low. For example, one or more of the thermal cameras in one or more of the disclosed embodiments may be based on a thermopile sensor configured to provide temperature measurements at a rate below at least one of the following rates: 15 Hz, 10 Hz, 5 Hz, and 1 Hz.

In some embodiments, the field of view of the thermal camera is limited by a field limiter. For example, the thermal camera may be based on a Texas Instruments TMP006B IR thermopile utilizing a field limiter made of thin polished metal, or based on Melexis MLX90614 IR thermometers in TO-39 package.

For a better understanding of some of the disclosed embodiments, and not because the following theoretical discussion is necessary to make and/or use the disclosed embodiments, the following non-limiting theoretical discussion describes why the accuracy of the object temperature change (ΔT) readings, over a certain duration appropriate for the specific application, is expected to often be better than the accuracy of the object temperature (T) readings when dealing with sensors that measure temperature, such as thermopiles or microbolometer. If the following theoretical discussion is found to be inaccurate, then it should be disregarded without limiting the scope of the disclosed embodiments in any way.

One problem with thermometers is that object temperature is hard to measure. Exact sensor output for a given object's temperature depends on properties of each particular sensing element, where each sensing element of the same sensor model may have its own operating parameters such as its own zero point, its own nonlinear coefficients, and/or its own electrical properties. Thus, one sensing element's operating parameters may be quite different from another's. However, when it comes to a small change in object temperature, such as from 35.7° C. to 35.9° C., then the zero point has a small impact when measuring difference between two readings, and the nonlinear effects are small since the difference itself is small. For example, although the uniformity of different Texas Instruments TMP006B infrared thermopile sensors is usually not observed, the response of each particular sensor is quite linear and stable, meaning that with proper calibration and filtering, it is possible to achieve a precision of temperature difference of 0.1° C., and even better, over a certain duration appropriate for a certain application.

Accuracy of a focal-plane array (FPA) of sensing elements may be given in terms of temperature measurement accuracy. For example, accuracy of 0.2° C. means that any sensing element in the FPA will provide the same ±0.2° C. temperature for a given object. However, when the current reading of a certain sensing element is compared to its previous readings (as opposed to the case where the current reading of the certain sensing element is compared to previous readings of other sensing elements), then the variability between the sensing elements essentially does not affect the accuracy of ΔT obtained from the certain sensing element. The Melexis MLX90621 16×4 thermopile array is an example of a thermopile based FPA that may be utilized by some of the disclosed embodiments, optionally with optics suitable for short distance. A FLIR Lepton® long-wave infrared camera module with an 80×60 microbolometer sensor array, weighing 0.55 g, is an example of a microbolometer based FPA that may be utilized by some of the disclosed embodiments, optionally with optics suitable for short distance.

The specific detectivity, noted as D*, of bolometers and thermopiles depends on the frequency of providing the temperature readings. In some embodiments, there is essentially no need for tracking and/or image registration, thus it is possible to configure the thermopile to provide temperature readings at rates such as 15 Hz, 10 Hz, 5 Hz, and even 1 Hz or lower. A thermopile with reaction time around 5-10 Hz may provide the same level of detectivity as a bolometer, as illustrated for example in the publication Dillner, U., Kessler, E., & Meyer, H. G. (2013), “Figures of merit of thermoelectric and bolometric thermal radiation sensors”, J. Sens. Sens. Syst, 2, 85-94. In some cases, operating at low frequencies provides benefits that cannot be achieved when there is a need to apply image registration and run a tracker, which may enable a reduction in price of the low frequency sensors that may be utilized.

In some embodiments of thermopiles, there are many thermocouples where one side of each couple is thermally connected to a measuring membrane, while another side is connected to the main body of the thermometer. In each thermocouple, a voltage dependent on temperature difference is generated according to Seebeck's effect. When these thermocouples are connected in series, the effect is multiplied by the number of thermocouples involved. For each thermocouple, the voltage generated is defined by Seebeck's formula: dV=S*dT, where dV is the generated voltage difference, dT is the temperature difference, and S is a Seebeck coefficient that is a material-dependent coefficient (for example 0.5 mV/K). Since accurate voltage measurement of several microvolts is achievable, this method may allow detection of ΔT at high resolution, such as 0.01 oK or less. That being said, since a thermocouple senses the difference between two ends and not the object temperature, it is required to know the temperature of the main thermometer body with high precision, otherwise the precision may drop. More information on Seebeck's effect and micromachined thermopiles can be found in the publication Graf, A., Arndt, M., & Gerlach, G. (2007), “Seebeck's effect in micromachined thermopiles for infrared detection. A review”, Proc. Estonian Acad. Sci. Eng, 13(4), 338-353.

In some embodiments of bolometers, the measuring membrane is connected to a material that changes its resistance significantly when the temperature is changed as follows: R=R₀*(1+a*dT), where R is resistance at a given temperature, and R₀ and ‘a’ are material-dependent parameters. In one example of vanadium pentoxide, the sensitivity highly depends on the layer creation technology, and the resistance change may be as high as 4% per Kelvin, where 2% may be a typical value. Since the resistance value depends on the temperature, the measurements are theoretically independent of the temperature of the main thermometer body. However, in practice, there may be a heat flow between the measuring membrane and the main body, which imposes a practical limit on the maximum temperature difference. In addition, the maximum temperature difference may not be the same in both negative and positive directions, with higher differences causing an increase in the measurement error.

Both bolometers and thermopiles work better when the object temperature is close to the detector temperature. Maintaining the temperature of the detector constant is helpful to detect small differences in object temperature precisely, thus, in some embodiments, the detectors are placed on a plate of metal having high thermal conductance, such as aluminum or copper, which optionally has Peltier elements and several high precision contact thermometers for temperature control.

Using several detectors instead of a single detector may decrease signal noise and increase stability. If the measurement electronics of a particular sensor has a long-term measurement drift (which may be added at on-chip circuit level), then using multiple sensors may be a practical way to remove the drift, such as in a small temperature-stabilized platform with several sensors.

One limitation to detecting differences in an object's temperature is often the ability to keep the sensors' temperature constant. At least with several relatively inexpensive commercially available sensors, temperature is measured with 0.01-0.02° C. steps, meaning that even a single sensor may be able to detect ΔT of 0.04° C. or less. However, for thermopile sensors, the detected signal is the difference between the object temperature and the thermometer case temperature, thus, the case temperature needs to be measured with the appropriate precision. In one example, such high precision measurements may be obtained utilizing high quality temperature stabilization of the thermometer's base metal plate, which may require several high-precision contact thermometers and Peltier elements to control the temperature. In another example, the thermal camera uses bolometers, which are not so sensitive to case temperature, and enable operation in room temperature as long as the environment is maintained with the bolometers' insensitivity range, such as ±3° C. changes.

The following is an additional and/or alternative description why the accuracy of the object temperature change (ΔT) readings, over a certain duration appropriate for the specific application, is expected to often be better than the accuracy of the object temperature (T) readings, when dealing with sensors that measure temperature, such as thermopiles. The measurement error of a thermal camera that measures temperature (such as a thermopile or a bolometer) is the difference between the measured temperature and the actual temperature at the ROI.

In some embodiments, the temperature measurement error may be considered to be composed of two components: random error in temperature measurement (ERR_{TR}) and systematic error in temperature measurement (ERR_{TS}). ERR_{TR} are errors in temperature measurement that lead to measurable values being inconsistent when repeated measurements of a constant ROI temperature are taken, and its effect may be reduced significantly when measurements are averaged. ERR_{TS} are introduced by an offset, gain and/or nonlinearity errors in the thermal camera, and its effect is not reduced significantly when measurements are averaged.

In many of the disclosed embodiments, inaccurate sensor calibration is expected to affect ERR_{TS} more than it affects ERR_{TR} (both when repeated measurements of a constant ROI temperature are taken and when repeated measurements of a changing ROI temperature are taken). Therefore, the novel embodiments of detecting a physiological response based on a temperature change at the ROI (ΔT_{ROI}) by a thermal camera that remains pointed at the ROI when the user's head makes angular movements—enable the system to utilize relatively inexpensive thermal sensors that could not be used for detecting the physiological response had the thermal camera not remained pointed at the ROI when the user's head makes angular movements.

In one embodiment, the thermal camera measures temperature at the ROI, and the system's nominal measurement error of the temperature at the ROI (T_{ROI}, ERR_{TPOI}) is at least twice the system's nominal measurement error of the temperature change at the ROI (ΔT_{ROI}, ERR_{ΔTROI}) when the user's head makes angular movements also above 0.1 rad/sec. Optionally, in this embodiment, the system is able to identify a physiological response, causing a temperature change at the ROI, which is below ERR_{TROI} and until ERR_{ΔTROI}.

In a variation of the previous embodiment, the thermal camera measures temperature at the ROI, and the system's nominal measurement error of the temperature at the ROI (TROT, ERR_{TROI}) is at least five the system's nominal measurement error of the temperature change at the ROI (ΔT_{ROI}, ERR_{ΔTROI}) when the user's head makes angular movements also above 0.5 rad/sec. Optionally, in this embodiment, the system is able to identify a physiological response, causing a temperature change at the ROI, which is below ERR_{TROI} and above ERR_{ΔTROI}.

The maximum rate of angular movement of the user's head in which ERR_{ΔTROI} is still significantly smaller than ERR_{TROI} may depend on the frame that mounts the system to the user. Sentences such as “when the user's head makes angular movements also above 0.1 rad/sec” refer to reasonable rates to which the frame/system is designed, and do not refer to situations where the frame/system is unstable. For example, a sport sunglasses frame equipped with a few small thermopile sensors is expected to stay stable also at head movements of 1 rad/sec, but most probably will generate measurement errors at head movements above 5 rad/sec.

Unless otherwise indicated, as a result of being physically coupled to the frame, the thermal camera remains pointed at the ROI when the user's head makes angular movements. Sentences such as “the thermal camera is physically coupled to the frame” refer to both direct physical coupling to the frame, which means that the thermal camera is fixed to/integrated into the frame, and indirect physical coupling to the frame, which means that the thermal camera is fixed to/integrated into an element that is physically coupled to the frame. In both the direct physical coupling and the indirect physical coupling embodiments, the thermal camera remains pointed at the ROI when the user's head makes angular movements. In some examples, the rate of angular movement referred to in sentences such as “when the user's head makes angular movements” is above 0.02 rad/sec, 0.1 rad/sec, 0.5 rad/sec, or 1 rad/sec.

In some embodiments, a processor is configured to identify a physiological response based on ΔT_{ROI} reaching a threshold. The threshold may include at least one of the following thresholds: threshold in the time domain, threshold in the frequency domain, an upper threshold where reaching the threshold means equal or above the threshold, and a lower threshold where reaching the threshold means equal or below the threshold. Herein, sentences such as “X reaching a threshold Y” are to be interpreted as X≧Y. For example, when the threshold equals 0.5, then both ΔT_{ROI}=0.5 and ΔTROI=0.7 are considered values of ΔT_{ROI} that reach the threshold, while ΔT_{ROI}=0.3 is not considered a value that reaches the threshold.

In some embodiments, the threshold for detecting the physiological response may be a function of the systematic and random errors, such as: threshold<0.8*ERR_{TS}, threshold<0.5*ERR_{TS}, threshold<0.2*ERR_{TS}, ERR_{TS}>0.1° C. and threshold<0.1° C., and/or ERR_{TS}>0.4° C. and threshold<0.2° C.

The measurement error of a thermal camera that measures temperature changes (such as a pyroelectric sensor) is the difference between the measured temperature change and the temperature change at the ROI. Examples of pyroelectric sensors that may be useful for at least some of the embodiments herein, optionally with some adaptations, include: (i) Excelitas Technologies analog pyroelectric non-contact sensor series, having one, two, four, or more elements; (ii) Excelitas Technologies DigiPyro® digital pyroelectric non-contact sensor series, having two, four, or more elements; and (ii) Murata Manufacturing Co., Ltd. dual type pyroelectric infrared sensor series, or Parallel Quad Type Pyroelectric Infrared Sensor Series.

In some of the embodiments described herein, the thermal camera is based on an uncooled thermal sensor. Herein, an uncooled thermal sensor refers to a sensor useful for measuring wavelengths longer than 2500 nm, which (i) operates at ambient temperature, or (ii) is stabilized at a temperature that is no more than ±20° C. from the ambient temperature. Optionally, one or more of the thermal cameras herein may be based on at least one of the following uncooled thermal sensors: a microbolometer sensor (which refers herein to any kind of bolometer sensor), a pyroelectric sensor, and a ferroelectric sensor. In other embodiments, one or more of the thermal cameras may be based on a cooled thermal sensor.

In some of the embodiments, the thermal camera is based on a thermopile sensor. The reference Pezzotti, G., Coppa, P., & Liberati, F. (2006), “Pyrometer at low radiation for measuring the forehead skin temperature”, Revista Facultad de Ingenieria Universidad de Antioquia, (38), 128-135 describes one example of measuring the forehead temperature with a thermopile that provides accuracy better than 0.2° C., without necessitating physical contact with the forehead, and with a working distance between 350 and 400 mm. The optics in this example involves a single aspherical mirror, which may, or may not, be necessary when the thermal camera is located just a few centimeters from the ROI.

For various purposes, thermal cameras may be positioned in certain locations, e.g., in order to be able to take measurements of a certain region of interest (ROIs). Optionally, in order to improve the measurement accuracy, a thermal camera may be located away from a specific region, such as being located outside of the exhale streams of the mouth and nostrils. Herein, sentences such as “located outside the exhale streams of the mouth and nostrils” means located outside most of the normally expected exhale stream of the mouth and located outside most of the normally expected exhale streams from the nostrils. The normally expected exhale streams are determined according to a normal human who breathes normally, when having a relaxed (neutral) face, and when the neck, jaw, and facial muscles are not stretched nor contracted. For example, a thermal camera is considered to be located outside the exhale streams from the nostrils when it is located to the right of the right nostril, and/or to the left of the left nostril, and/or outside a 3D rectangle that extends from below the tip of the nose to the lower part of the chin with a base size of at least 4×4 cm. In another example, a thermal camera is considered to be located outside the exhale stream of the mouth when it is located outside a horizontal cylinder having height of 10-20 cm and diameter of 4-10 cm, where the top of the cylinder touches the base of the nose.

In the case of a thermal camera based on a thermal sensor such as a thermopile, the thermopile's reference junctions may compensate for changes in the temperature of the ROI. If the reference junction temperature is fixed, for example by placing the reference junctions over a heat sink and/or insulating them, then exhale streams from the nostrils and/or mouth may not affect the temperature difference between the ROI and the sensing junctions. However, when the reference junction temperature is not fixed, then the breath passing over the sensor may change the measured value of the thermopile merely because the temperature of the exhale stream is close to body temperature. For example, if the thermopile was at room temperature and the temperature of the reference junctions is essentially fixed, then the thermopile would register a voltage that is proportional to a change to the temperature between ROI and room temperature. However, if the sensing junctions are exposed to the exhale stream, then the thermopile may measure a wrong temperature of the ROI. In order to avoid such an error, in some embodiments, a non-well isolated thermal camera is located outside the exhale streams, which means that the thermal camera is not placed in front of the nostrils and/or in front of the mouth, but to the side, above, below, and/or in any other possible location that is away from the nostrils and the mouth. In some embodiments, another thermal camera may be located inside the exhale streams from at least one of the mouth and the nostrils. Additionally, some embodiments may further include another thermal camera located inside the exhale streams from at least one of the mouth and the nostrils.

In one embodiment, the system includes at least two thermal camera physically coupled to the frame and pointed at first and second ROIs (ROI₁ and ROI₂, respectively). The processor is configured to calculate ΔT_{ROI1} and ΔT_{ROI2} based on the temperature measurements of the first and second thermal cameras, and to identify the physiological response based on a difference between ΔTROI and ΔT_{ROI2}.

For example, assuming the physiological response is allergic reaction, ROI₁ is the nasal area, and ROI₂ is the forehead; when both ΔT_{ROI1} and ΔT_{ROI2} increase in 1° C. then it is less probable that the cause is allergic reaction compared to a case where ΔT_{ROI1} increases in 1° C. while ΔT_{ROI2} stays essentially the same. In another example, assuming the physiological response is allergic reaction, ROI₁ is the right side of the nasal area, and ROI₂ is the left side of the nasal area; when both ΔT_{ROI1} and ΔT_{ROI2} increase in 0.5° C. then it is more probable that the cause is allergic reaction compared to a case where ΔT_{ROI1} increases in 0.5° C. while ΔT_{ROI2} stays essentially the same. In still another example, assuming the physiological response is stress, ROI₁ is the nose, and ROI₂ is the maxillary; when both ΔT_{ROI1} and ΔT_{ROI2} decrease more than 0.2° C. then it is more probable that the cause is stress compared to a case where (ΔT_{ROI1} decreases more than 0.2° C. while ΔT_{ROI2} stays essentially the same.

Additional Considerations

FIG. 9a and FIG. 9b are schematic illustrations of possible embodiments for computers (400, 410) that are able to realize one or more of the embodiments discussed herein. The computer (400, 410) may be implemented in various ways, such as, but not limited to, a server, a client, a personal computer, a set-top box (STB), a network device, a handheld device (e.g., a smartphone), computing devices embedded in wearable devices (e.g., a smartwatch or a computer embedded in clothing), computing devices implanted in the human body, and/or any other computer form capable of executing a set of computer instructions. Further, references to a computer include any collection of one or more computers that individually or jointly execute one or more sets of computer instructions to perform any one or more of the disclosed embodiments.

The computer 400 includes one or more of the following components: processor 401, memory 402, computer readable medium 403, user interface 404, communication interface 405, and bus 406. In one example, the processor 401 may include one or more of the following components: a general-purpose processing device, a microprocessor, a central processing unit, a complex instruction set computing (CISC) microprocessor, a reduced instruction set computing (RISC) microprocessor, a very long instruction word (VLIW) microprocessor, a special-purpose processing device, an application specific integrated circuit (ASIC), a field programmable gate array (FPGA), a digital signal processor (DSP), a distributed processing entity, and/or a network processor. Continuing the example, the memory 402 may include one or more of the following memory components: CPU cache, main memory, read-only memory (ROM), dynamic random access memory (DRAM) such as synchronous DRAM (SDRAM), flash memory, static random access memory (SRAM), and/or a data storage device. The processor 401 and the one or more memory components may communicate with each other via a bus, such as bus 406.

The computer 410 includes one or more of the following components: processor 411, memory 412, and communication interface 413. In one example, the processor 411 may include one or more of the following components: a general-purpose processing device, a microprocessor, a central processing unit, a complex instruction set computing (CISC) microprocessor, a reduced instruction set computing (RISC) microprocessor, a very long instruction word (VLIW) microprocessor, a special-purpose processing device, an application specific integrated circuit (ASIC), a field programmable gate array (FPGA), a digital signal processor (DSP), a distributed processing entity, and/or a network processor. Continuing the example, the memory 412 may include one or more of the following memory components: CPU cache, main memory, read-only memory (ROM), dynamic random access memory (DRAM) such as synchronous DRAM (SDRAM), flash memory, static random access memory (SRAM), and/or a data storage device

Still continuing the examples, the communication interface (405,413) may include one or more components for connecting to one or more of the following: LAN, Ethernet, intranet, the Internet, a fiber communication network, a wired communication network, and/or a wireless communication network. Optionally, the communication interface (405,413) is used to connect with the network 408. Additionally or alternatively, the communication interface 405 may be used to connect to other networks and/or other communication interfaces. Still continuing the example, the user interface 404 may include one or more of the following components: (i) an image generation device, such as a video display, an augmented reality system, a virtual reality system, and/or a mixed reality system, (ii) an audio generation device, such as one or more speakers, (iii) an input device, such as a keyboard, a mouse, a gesture based input device that may be active or passive, and/or a brain-computer interface.

Functionality of various embodiments may be implemented in hardware, software, firmware, or any combination thereof. If implemented at least in part in software, implementing the functionality may involve a computer program that includes one or more instructions or code stored or transmitted on a computer-readable medium and executed by one or more processors. Computer-readable media may include computer-readable storage media, which corresponds to a tangible medium such as data storage media, or communication media including any medium that facilitates transfer of a computer program from one place to another. Computer-readable medium may be any media that can be accessed by one or more computers to retrieve instructions, code and/or data structures for implementation of the described embodiments. A computer program product may include a computer-readable medium.

In one example, the computer-readable medium 403 may include one or more of the following: RAM, ROM, EEPROM, optical storage, magnetic storage, biologic storage, flash memory, or any other medium that can store computer readable data. Additionally, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of a medium. It should be understood, however, that computer-readable medium does not include connections, carrier waves, signals, or other transient media, but are instead directed to non-transient, tangible storage media.

A computer program (also known as a program, software, software application, script, program code, or code) can be written in any form of programming language, including compiled or interpreted languages, declarative or procedural languages. The program can be deployed in any form, including as a standalone program or as a module, component, subroutine, object, or another unit suitable for use in a computing environment. A computer program may correspond to a file in a file system, may be stored in a portion of a file that holds other programs or data, and/or may be stored in one or more files that may be dedicated to the program. A computer program may be deployed to be executed on one or more computers that are located at one or more sites that may be interconnected by a communication network.

Computer-readable medium may include a single medium and/or multiple media (e.g., a centralized or distributed database, and/or associated caches and servers) that store the one or more sets of instructions. In various embodiments, a computer program, and/or portions of a computer program, may be stored on a non-transitory computer-readable medium. The non-transitory computer-readable medium may be implemented, for example, via one or more of a volatile computer memory, a non-volatile memory, a hard drive, a flash drive, a magnetic data storage, an optical data storage, and/or any other type of tangible computer memory to be invented that is not transitory signals per se. The computer program may be updated on the non-transitory computer-readable medium and/or downloaded to the non-transitory computer-readable medium via a communication network such as the Internet. Optionally, the computer program may be downloaded from a central repository such as Apple App Store and/or Google Play. Optionally, the computer program may be downloaded from a repository such as an open source and/or community run repository (e.g., GitHub).

At least some of the methods described in this disclosure, which may also be referred to as “computer-implemented methods”, are implemented on a computer, such as the computer (400,410). When implementing a method from among the at least some of the methods, at least some of the steps belonging to the method are performed by the processor (401,411) by executing instructions. Additionally, at least some of the instructions for running methods described in this disclosure and/or for implementing systems described in this disclosure may be stored on a non-transitory computer-readable medium.

As used herein, references to “one embodiment” (and its variations) mean that the feature being referred to may be included in at least one embodiment of the invention. Moreover, separate references to “one embodiment”, “some embodiments”, “another embodiment”, and “still another embodiment”, etc., may refer to the same embodiment, may illustrate different aspects of an embodiment, and/or may refer to different embodiments.

Some embodiments may be described using the verb “indicating”, the adjective “indicative”, and/or using variations thereof. For example, a value may be described as being “indicative” of something. When a value is indicative of something, this means that the value directly describes the something and/or is likely to be interpreted as meaning that something (e.g., by a person and/or software that processes the value). Verbs of the form “indicating” or “indicate” may have an active and/or passive meaning, depending on the context. For example, when a module indicates something, that meaning may correspond to providing information by directly stating the something and/or providing information that is likely to be interpreted (e.g., by a human or software) to mean the something. In another example, a value may be referred to as indicating something, in this case, the verb “indicate” has a passive meaning: examination of the value would lead to the conclusion to which it indicates.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

In addition, use of the “a” or “an” is employed to describe one or more elements/components/steps/modules/things of some of the embodiments herein. This description should be read to include one or at least one, and the singular also includes the plural unless it is obvious that it is meant otherwise. Additionally, the phrase “based on” is intended to mean “based, at least in part, on”.

While the methods disclosed herein may be described and shown with reference to particular steps performed in a particular order, it is understood that these steps may be combined, sub-divided, and/or reordered to form an equivalent method without departing from the teachings of some of the embodiments. Accordingly, unless specifically indicated herein, the order and grouping of the steps is not a limitation of the embodiments. Furthermore, methods and mechanisms of some of the embodiments will sometimes be described in singular form for clarity. However, some embodiments may include multiple iterations of a method or multiple instantiations of a mechanism unless noted otherwise. For example, when a processor is disclosed in one embodiment, the scope of the embodiment is intended to also cover the use of multiple processors. Certain features of some of the embodiments, which may have been, for clarity, described in the context of separate embodiments, may also be provided in various combinations in a single embodiment. Conversely, various features of some of the embodiments, which may have been, for brevity, described in the context of a single embodiment, may also be provided separately or in any suitable sub-combination.

Embodiments described in conjunction with specific examples are presented by way of example, and not limitation. Moreover, it is evident that many alternatives, modifications, and variations will be apparent to those skilled in the art. It is to be understood that other embodiments may be utilized and structural changes may be made without departing from the scope of the appended claims and their equivalents.

What is claimed is:

1. A system configured to detect an allergic reaction of a user, comprising:
a frame configured to be worn on the user's head;
a thermal camera, weighing less than 5 g, physically coupled to the frame, located less than 10 cm away from the user's face, and configured to take thermal measurements of at least part of the user's nose (TH_{N}); and
a circuit configured to determine an extent of the allergic reaction based on TH_{N}.

2. The system of claim 1, wherein the allergic reaction is selected from among the following reactions: allergic rhinitis, atopic dermatitis, and anaphylaxis.

3. The system of claim 1, wherein the allergic reaction is a reaction to one or more of the following allergens: pollen, dust, latex, perfume, a drug, peanuts, eggs, wheat, milk, and seafood.

4. The system of claim 1, wherein the thermal camera is based on at least one of the following uncooled sensors: a thermopile, a pyroelectric, and a microbolometer.

5. The system of claim 4, wherein the thermal camera provides temperature measurement accuracy better than ±1.0° C. and provides temperature change (ΔT) accuracy better than ±0.10° C.

6. The system of claim 1, further comprising a second thermal camera, weighing less than 5 g, physically coupled to the frame, located less than 10 cm away from the user's face, and configured to take second thermal measurements of at least part of the user's mouth; wherein the circuit is further configured to utilize the second thermal measurements to determine the extent of the allergic reaction.

7. The system of claim 1, wherein the circuit is further configured to detect a rise in nasal temperature and alert the user of the allergic reaction.

8. The system of claim 7, wherein the rise involves an increase of at least 0.8° C. within less than 30 minutes.

9. The system of claim 7, wherein the rise involves an increase of at least 0.5° C. within less than 10 minutes.

10. The system of claim 1, wherein the circuit is further configured to identify a potential allergen by estimating a time of exposure to the allergen from a graph exhibiting deviation over time of mean nasal temperature from baseline, and analyzing items to which the user was exposed at that time in order to identify the potential allergen.

11. The system of claim 1, wherein the thermal camera is not in physical contact with the nose, and remains pointed at the nose when the user's head makes angular movements also above 0.1 rad/sec.

12. A method for detecting an allergic reaction of a user, comprising:
receiving, by a system comprising a circuit, thermal measurements of at least part of the user's nose (TH_{N}); wherein the measurements are taken by a thermal camera weighing less than 5 g, which is physically coupled to the frame worn on the user's head and is located less than 10 cm away from the user's face;
determining, based on TH_{N}, whether there is an increase in temperature in the nasal region of the user reaches a threshold; and
responsive to a determination that the increase in temperature in the nasal region of the user reaches the threshold, generating an indication indicative of an onset of an allergic reaction of the user.

13. The method of claim 12, wherein the threshold corresponds to an increase of at least 0.5° C., and further comprising generating the indication indicative of the onset of the allergic reaction of the user responsive to determining that the increase occurred during a period of time that is shorter than 10 minutes.

14. The method of claim 12, wherein the threshold corresponds to an increase of at least 0.8° C., and further comprising generating the indication indicative of the onset of the allergic reaction of the user responsive to determining that the increase occurred during a period of time that is shorter than 30 minutes.

15. The method of claim 12, further comprising determining an extent of the allergic reaction based on at least one of the following values: a magnitude of the increase in the temperature in the nasal region, a rate of the increase to the temperature in the nasal region, a duration within which the threshold was reached.

16. The method of claim 12, further comprising identifying a potential allergen by estimating a time of exposure to the allergen from a graph exhibiting deviation over time of mean nasal temperature from baseline, and analyzing items to which the user was exposed at that time in order to identify the potential allergen.

17. The method of claim 16, further comprising utilizing an image taken by a camera mounted to the frame in order to display the potential allergen to the user.

18. A non-transitory computer-readable medium having instructions stored thereon that, in response to execution by a system including a processor and memory, causes the system to perform operations comprising:
receiving, by a system comprising a circuit, thermal measurements of at least part of the user's nose (TH_{N}); wherein the measurements are taken by a thermal camera weighing less than 5 g, which is physically coupled to the frame worn on the user's head and is located less than 10 cm away from the user's face;
determining, based on TH_{N}, whether there is an increase in temperature in the nasal region of the user reaches a threshold; and
responsive to a determination that the increase in temperature in the nasal region of the user reaches the threshold, generating an indication indicative of an onset of an allergic reaction of the user.

19. The non-transitory computer-readable medium of claim 18, wherein the threshold corresponds to an increase of at least 0.8° C., and further comprising instructions defining a step of generating the indication indicative of the onset of the allergic reaction of the user responsive to determining that the increase occurred during a period of time that is shorter than 30 minutes.

20. The non-transitory computer-readable medium of claim 18, further comprising instructions defining a step of determining an extent of the allergic reaction based on at least one of the following values: a magnitude of the increase in the temperature in the nasal region, a rate of the increase to the temperature in the nasal region, a duration within which the threshold was reached.
